(19) United States
(12) Patent Application Publication
(10) Pub. No.: US 2012/0252896 A1
(43) Pub. Date: Oct. 4, 2012

(54) METHODS AND COMPOSITIONS FOR MODULATION OF T-CELLS VIA THE KYNURENINE PATHWAY

(76) Inventors: Joel D. Ernst, New York, NY (US); Ludovic Desvignes, New York, NY (US)

(21) Appl. No.: 13/510,655

(22) PCT Filed: Nov. 19, 2010
(86) PCT No.: PCT/US10/03004

§ 371 Date: Jun. 18, 2012

Related U.S. Application Data
(60) Provisional application No. 61/281,683, filed on Nov. 20, 2009.
(60) Provisional application No. 61/267,653, filed on Dec. 8, 2009.

(51) Int. Cl. A61K 31/196; C12Q 1/02; A61P 37/06; A61K 31/198; A61P 29/00; C12N 5/07; G01N 33/53

(52) U.S. Cl. 514/564; 435/375; 435/29; 435/7.92; 514/567

(57) ABSTRACT

The present invention provides methods for the modulation of T cells and T cell responses, particularly of Th17 effector cells. The invention provides methods of and compositions for modulating T cells, particularly T cells expressing IL-17, particularly Th17 effector cells, via the tryptophan metabolism pathway, particularly using tryptophan metabolites, kynurenines and kynurenine analogs or metabolites. The invention provides assays for screening Th17 modulators and kynurenine analogs or compounds.

### GOVERNMENTAL SUPPORT

The research leading to the present invention was supported, at least in part, by a grant from The U.S. National Institutes of Health, National Institute of Allergy and Infectious Diseases, Grant Nos. R03 A1053074 and R01 AI059667. Accordingly, the Government may have certain rights in the invention.

### FIELD OF THE INVENTION

The present invention relates generally to the modulation and manipulation of T cells and T cell responses, particularly of IL-17 expressing cells, particularly of Th17 effector cells. The invention further relates to methods of and compositions for modulating T cells, particularly IL-17 expressing cells, particularly Th17 effector cells, via the tryptophan metabolism pathway, particularly using tryptophan metabolites, kynurenines and kynurenine analogs or metabolites. The invention includes assays for screening Th17 modulators and kynurenine analogs or compounds.

### BACKGROUND OF THE INVENTION

The interferon gamma-inducible enzyme, Indoleamine-2,3-dioxygenase metabolizes the aromatic amino acid tryptophan, to a series of products collectively termed kynurenines. These include formylkynurenine, kyurenine, 3-hydroxykynurenine, 3-hydroxyanthranic acid, and quinolinate. The tryptophan metabolism pathway and its metabolite structures are depicted in FIG. 1. Diverse biological activities have been attributed to certain of the kynurenines, including neurotoxicity and immunomodulatory effects.

The kynurenine pathway is the main pathway for tryptophan metabolism. It generates compounds that can modulate activity at glutamate receptors and possibly nicotinic receptors, in addition to some as-yet-unidentified sites. The pathway is in a unique position to regulate other aspects of the metabolism of tryptophan to neuroactive compounds, and also seems to be a key factor in the communication between the nervous and immune systems. It also has potentially important roles in the regulation of cell proliferation and tissue function in the periphery. As a result, the pathway presents a multitude of potential sites for drug discovery in neuroscience, oncology and visceral pathology

T helper 17 cells (Th17) are a recently identified subset of T helper cells producing interleukin 17. They are considered developmentally distinct from Th1 and Th2 cells and excessive amounts of Th17 cell are thought to play a key role in autoimmune diseases such as Multiple Sclerosis (which was previously thought to be caused by Th1 cells), rheumatoid arthritis, and Crohn's Disease (Harrington L E, et al. (2005) Nat. Immunol. 6 (11): 1123-32; Stockinger B, Veldhoen M (2007) Curr. Opin. Immunol. 19 (3): 281-6). Th 17 cells are thought to play a role in inflammation and tissue injury in these conditions (Steinman L (2007) Nat. Med. 13 (2): 139-45). Tₕ17 cells have been broadly implicated in autoimmune disease and Tₕ17 cells have been shown to be highly pathogenic. Other studies of Tₕ17 cells have demonstrated preferential induction of IL-17 in cases of host infection with various bacterial and fungal species. Th17 cells can cause severe autoimmune diseases, however they do serve an important function in anti-microbial immunity at epithelial/mucosal barriers, producing cytokines (such as interleukin 22) which stimulates epithelial cells to produce anti-microbial proteins to clear certain types of microbes (such as Candida and Staph). Th17 cells have been recently demonstrated to have a role in cancer immunity, including whereby tumor specific Th17 cells prevented melanoma lung tumor development in cancer models (Martin-Orozco, N et al (2009) Immunity 31(5):787-798). Which cytokines exactly contribute to Th17 formation are still being determined, however transforming growth factor beta (TGF-β), interleukin 6 (IL-6), interleukin 21 (IL-21) and interleukin 23 (IL-23) have been implicated in mice and humans (Dong C (2008) Nat. Rev. Immunol. 8 (5): 337-48; Manel N, Unutmaz D, Littman DR (2008) Nat. Immunol. 9 (6): 641-9). Other proteins involved in Th17 cell differentiation are signal transducer and activator of transcription 3 (STAT3) and the retinoic-acid-receptor-related orphan receptors alpha (RORα) and RORγ (Dong C (2008) Nat. Rev. Immunol. 8 (5): 337-48) Effector cytokines associated with this cell type are IL-17, IL-21 and IL-22 (Ouyang W, Kolls J K, Zheng Y (2008) Immunity 28 (4): 454-67).

Despite an increased understanding and knowledge of immune system cells and responses, a need still exists for the particular and directed modulation of T cells, particularly of T cell subsets, such as T helper cells and Th17 cells. The availability of agents or assays for agents to specifically modulate Th17 cells, including to modulate the activation and/or differentiation of Th17 cells, would have potential clinical impact and application in various diseases and conditions, such as in immune and inflammatory conditions, particularly wherein Th17 cell activation or proliferation is involved in the etiology of the disease. Therefore, in view of the aforementioned deficiencies attendant with prior art methods of modulation of inflammatory or autoimmune disorders, it should be apparent that there still exists a need in the art for methods and agents to specifically alter Th17 cell response and activity.

The citation of references herein shall not be construed as an admission that such is prior art to the present invention.

### SUMMARY OF THE INVENTION

The present invention relates generally to the modulation of T cells, and particularly to the modulation of Th17 cells and their associated activities or cytokines, particularly IL-17 and IL-23. The present invention describes that tryptophan metabolites, kynurenines, provided as a mixture, or as individual compounds, specifically modulate IL-17 expressing cells, particularly Th17 cells. Tryptophan metabolites, kynurenines, provided as a mixture, or as individual compounds, inhibit differentiation of naive T lymphocytes to Th17 effector cells, with effects on Th1 differentiation only at higher (i.e., 5-fold) concentrations. The invention provides kynurenines, particularly L-kynurenine, 3-hydroxy-DL-kynurenine, and 3-hydroxyanthranilic acid, as modulators of cells expressing IL-17 and their activity, including for the inhibition of IL-17 production. The invention provides kynurenines, particularly L-kynurenine, 3-hydroxy-DL-kynurenine, and 3-hydroxyanthranilic acid, as modulators of Th17 cells and their activity, including for the inhibition of IL-17 production and the inhibition of IL-23 action.

Modulation of T lymphocyte differentiation, particularly of IL-17 expressing cells including Th17 cells, has multiple potential applications, including for treatment of inflammatory diseases, including autoimmune diseases and inflammation associated with acute and chronic infectious diseases. In addition, modulation of T lymphocyte differentiation in conjunction with vaccination has application in enhancing the efficacy of certain vaccines, and may prevent adverse effects of certain vaccines. Inhibition of Th17 cell activation or activity has application in reducing or alleviating inflammation, inflammatory conditions and auto-immune disorders or conditions. Stimulation of Th17 cells has implications in and potential application for cancer immunity, cancer therapy, and antimicrobial immunity and clearance of microbes or fungi.

As lead compounds, kynurenines or kynurenine analogs provide anti-inflammatory and/or immunomodulatory drugs or compounds. In addition, they have use and application in further characterizing the molecular mechanisms of inhibition of Th17 differentiation and in the discovery, screening and development of analogs with enhanced potency and efficacy in treatment and modulation of disease and the immune response.

Kynurenine antagonists or inhibitors of tryptophan metabolic pathway enzymes, such as IDO, may be utilized in stimulating or activating IL-17 expressing cells. Kynurenine antagonists or inhibitors of tryptophan metabolic pathway enzymes, such as IDO, may be utilized in stimulating or activating Th17 cells. Activation or stimulation of Th17 cells has uses and application in cancer immunity, cancer therapy and microbial immunity and clearance.

In accordance with the present invention, a method is provided for modulation of T cell, particularly including Th17 cell, differentiation, activation, and/or activity by administration of one or more tryptophan metabolite, kynurenine or kynurenine analog or by administration of a tryptophan pathway inhibitor, an inhibitor or antagonist of the tryptophan metabolic pathway or the kynurenine pathway, or a kynurenine analog antagonist. In accordance with the present invention, a method is further provided for inhibition of Th17 cell differentiation, activation, and/or activity by administration of one or more tryptophan metabolite, kynurenine or kynurenine analog. In accordance with the present invention, a method is further provided for inhibition of differentiation, activation, and/or activity of IL-17 expressing cells by administration of one or more tryptophan metabolite, kynurenine or kynurenine analog.

The concept of the kynurenine pathway and kynurenine metabolites specifically modulating Th17 cells, and modulating IL-17 and IL-23 activity, contemplates that kynurenines act as antagonists of Th17 activity and/or differentiation. It is the specificity of kynurenines in modulating Th17 cells and the particular alteration of Th17 cells and 11-17 and these effects in helper T cell activity and Th17 function that offer the promise of a broad spectrum of diagnostic and therapeutic utilities, including in inflammation and immune cell function.

The invention thus provides kynurenines, including one or more of L-kynurenine, 3-hydroxy-DL-kynurenine, and 3-hydroxyanthranilic acid, as modulators of IL-17 expressing cells and their activity. The invention thus provides kynurenines, including one or more of L-kynurenine, 3-hydroxy-DL-kynurenine, and 3-hydroxyanthranilic acid, as modulators of Th17 cells and their activity. The invention contemplates the use and application of kynurenine analogs, including natural metabolite analogs and synthetically or chemically generated analogs in the compositions and methods of the invention.

The invention includes an assay system for screening of potential drugs, including kynurenine analogs and antagonists, effective to modulate the activity of or differentiation of Th17 cells, the expression or production of IL-17, and/or the action of IL-23. The invention includes an assay system for screening for modulators of IDO in target mammalian cells, which enzyme converts tryptophan to kynurenines. In one instance, the test drug can be administered to a cellular sample, particularly a sample comprising T cells, particularly including or consisting of Th17 cells, in the presence or absence of a Th17 cell stimulator, to determine its effect upon the activity of Th17 cells, including determining the levels of 11-17 or of Th17 cell mediated cellular response, by comparison with a control.

The assay system could more importantly be adapted to identify drugs or other entities that are capable of specifically modulating Th17, thereby potentiating or inhibiting Th17 cell activity or Th17 cell factor expression, such as 11-17. Such assay would be useful in the development of drugs that would be specific against Th17 particular cellular activity, or that would potentiate such activity, in time or in level of activity. For example, such drugs might be used to alleviate inflammation, detrimental immune response or auto-immune disorders or conditions, or to treat other pathologies, as for example, in making a more potent or specific immune system modulator.

In yet a further embodiment, the invention contemplates antagonists of the activity of a kynurenine, effective to activate or stimulate Th17 cells. In yet a further embodiment, the invention contemplates antagonists of the activity of a kynurenine, effective to activate or stimulate IL-17 expressing cells. In particular, an agent or molecule that inhibits IDO or tryptophan metabolism or otherwise antagonizes kynurenines or their activity.

The diagnostic utility of the present invention extends to the use of kynurenines in assays to screen for or assess Th17 cell activity or Th17 cell-mediated responses or in characterizing an inflammatory or immune system/immune cell response. The relevance or prevalence of Th17 cells can be determined or assessed by determining alterations, for example, in IL-17 levels in response to or in the presence of kynurenines. The diagnostic utility of the present invention extends to the use of kynurenines in assays to screen for or assess IL-17 expressing cell activity or IL-17 expressing cell-mediated responses or in characterizing an inflammatory or immune system/immune cell response.

Thus, kynurenines and/or analogs thereof, and any antagonists or antibodies that may be raised thereto, are capable of use in connection with various diagnostic techniques, including immunoassays, such as a radioimmunoassay, using for example, an antibody to Th17 cells or to IL-17 or other immune cell factors that has been labeled by either radioactive addition, or radioiodination.

In an immunoassay, a control quantity of the antagonists or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

The present invention includes an assay system which may be prepared in the form of a test kit for the quantitative analysis of the extent of the presence or activity of IL-17 expressing cells, or to identify drugs or other agents that may mimic or block their activity or kynurenine activity. The present invention includes an assay system which may be prepared in the form of a test kit for the quantitative analysis of the extent of the presence or activity of T cells (including Th17 cells), or to identify drugs or other agents that may mimic or block their activity or kynurenine activity. The system or test kit may comprise a labeled component prepared by one of the radioactive and/or enzymatic techniques discussed herein, coupling a label to kynurenine(s), their agonists and/or antagonists, or to the Th17 cells and one or more additional immunochemical reagents, at least one of which is a free or immobilized ligand, capable either of binding with the labeled component, its binding partner, one of the components to be determined or their binding partner(s).

In a further embodiment, the present invention relates to certain therapeutic methods which would be based upon the activity of the kynurenine(s) or their analogs, or upon agents or other drugs determined to possess the same activity. A first therapeutic method is associated with the prevention of the manifestations of inflammatory or immune system/immune cell conditions causally related to or following from the activity of Th17 cells or their cellular factors, and comprises administering an agent capable of modulating the production and/or activity of the Th17, such as kynurenines, either individually or in mixture with each other, in an amount effective to prevent the development of or alleviate the symptoms of those conditions or cellular pathology associated with those conditions in the host. For example, kynurenines, their analog(s) or drugs having like activity, or their antagonists or drugs blocking their activity, may be administered to inhibit or potentiate Th17 activity, as in the inhibition of an imflammatory or immune system response or in the potentiation of cancer immunity or microbial clearance.

More specifically, the therapeutic method generally referred to herein includes a method for the treatment of various pathologies or other cellular dysfunctions and derangements by the administration of pharmaceutical compositions that may comprise kynurenines or analogs or antagonists thereof, effective inhibitors or enhancers of activation of Th17 cells, or other equally effective drugs developed for instance by a drug screening assay prepared and used in accordance with a further aspect of the present invention. For example, kynurenines, including one or more of L-kynurenine, 3-hydroxy-DL-kynurenine, and 3-hydroxyanthranilic acid or analogs or mimetics thereof, may be administered to alleviate inflammation or immune system response by inhibiting Th17 cell differentiation and/or activity.

It is a further object of the present invention to provide a method and associated assay system for screening substances such as drugs, agents and the like, potentially effective in either mimicking the activity of kynurenines or combating the adverse effects of Th17 cells and/or of IL-17 activity.

It is a still further object of the present invention to provide a method for the treatment of mammals to control the amount or activity of Th17 cells so as to alter the adverse consequences of such presence or activity, or where beneficial, to enhance such activity. Thus, inhibition of Th17 via the kynurenine pathway may reduce inflammation and/or detrimental immune system response(s). Conversely, activation of Th17 by blocking or antagonizing the kynurenine pathway may serve to enhance cancer immunity, alleviate tumors or cancer cells, and clear or otherwise reduce infectious agent microbes.

It is a still further object of the present invention to provide a method for the treatment of mammals to specifically control or modulate the amount or activity of Th17 cells, so as to treat or avert the adverse consequences of immune, inflammatory or idiopathic pathological states.

It is a still further object of the present invention to provide pharmaceutical compositions for use in therapeutic methods which comprise or are based upon the kynurenine pathway and tryptophan metabolites, or upon agents or drugs that control the production, or that mimic or antagonize the activities thereof.

Other objects and advantages will become apparent to those skilled in the art from a review of the following description which proceeds with reference to the following illustrative drawings.

### BRIEF DESCRIPTION OF THE DRAWINGS

FIG. 1 depicts the metabolic pathway of tryptohan metabolism, including identification and structure of its various metabolites and kynurenines.

FIG. 2. Flow cytometry analysis of the chimerism in lung leukocyte population of mice 6 weeks after-irradiation and injection of CD45.1+IFN R1+/+ bone marrow stem cells in CD45.1+IFN R1+/+ (W W) or CD45.2+IFN R1−/− (W K) mice or CD45.2+IFN R1−/− bone marrow stem cells in CD45.1+IFN R1+/+ (K W) or CD45.2+IFN R1−/− (K K) mice.

FIG. 3. Control of Long-Term M. tuberculosis Infection is Impaired in the Absence of IFN R1 on Nonhematopoietic Cells.

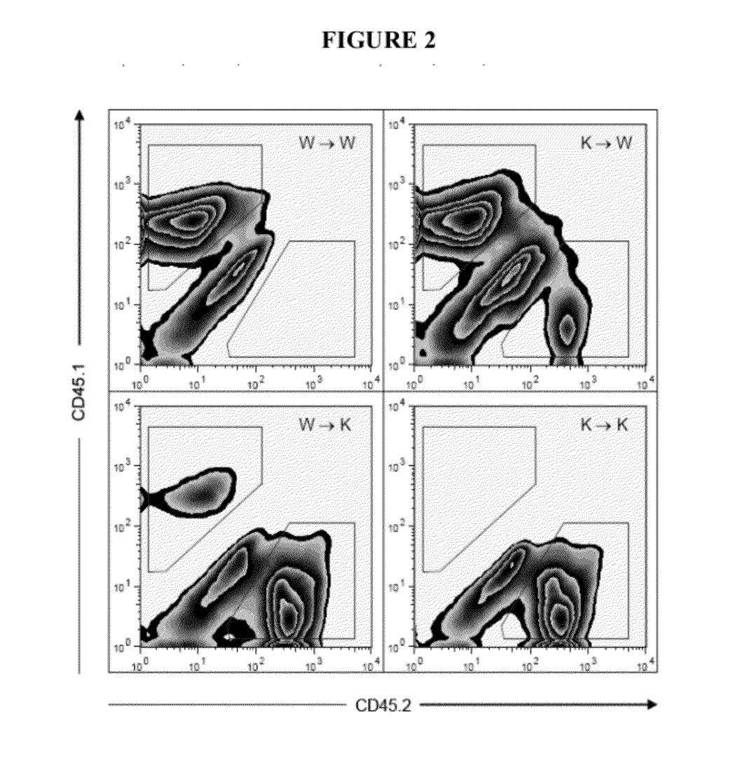

(A) Survival of chimeric mice after infection with M. tuberculosis: W W (n=5), W K (n=15), K W (n=15), K K (n=10). Data are representative of three independent experiments. Groups were compared using a log rank test. (B) Bacterial load in the lungs of infected chimeric mice as evaluated by plating serial dilutions of lung homogenates on 7H11 agar. Data are representative of three independent experiments and expressed as the mean (±S.E.) of 4 mice per time point and per group. Groups were compared using unpaired Student's t test with a 95% confidence interval. **p<0.01, ***p<0.005. Bacterial loads in the lungs of the respective groups of chimeric mice 3 and 4 weeks post-infection are presented in Supplementary FIG. 2A. (C) Lung gross pathology and histopathology in W W and W K mice 14 weeks post-infection with M. tuberculosis. Lung left lobes were fixed in paraformaldehyde for a minimum of 7 days. Histopathology was analyzed by hematoxylin and eosin (H&E) staining of paraformaldehyde-fixed paraffin-embedded 5 mm tissue sections 14 weeks after aerosol infection with M. tuberculosis. Original magnification, 40×. (D) Neutrophils (arrowhead) infiltrating the lungs of W K mice as evidenced by H&E staining 14 weeks post-infection. Original magnification, ×40. Insert magnification, ×1000.

(E) Kinyoun's acid-fast staining with brilliant green counterstaining showing neutrophils infected with M. tuberculosis (arrowheads) in the lungs of W K mice 14 weeks post-infection. Original magnification, ×40. Insert magnification, ×1000.

FIG. 4. Bacterial load in the lungs (A), mediastinal lymph node (MLN) (B) and spleen (C) of M. tuberculosis infected chimeric mice as evaluated by plating serial dilutions of lung homogenates on 7H11 agar. Data are expressed as the mean (±S.E.) of 4 mice per group at day 21 and 28 post-infection (A), per time point and per group (B) or represent individual measures and mean value for each group at day 97 post infection (B). Groups were compared using unpaired Student's t test with a 95% confidence interval.

**p<0.01.

FIG. 5. Lung gross pathology (A) and histopathology (B) in chimeric mice 28 days after aerosol infection with M. tuberculosis. Histopathology was examined on hematoxylin and eosin (H&E) stainings of paraformaldehyde fixed paraffin-embedded 5 μm thick tissue sections. Original magnification, 40×.

FIG. 6. Cell Populations in the Lungs of IFN R Chimeric Mice During M. tuberculosis Infection.

(A) Total cell number in the lungs and their viability were assessed on single cell suspensions obtained from infected chimeric mice. Viability was higher than 90%. Results are expressed as the mean number (±S.E.) of total cells per lung and for 4 mice per time point and per group. Data were compared using a two-tailed Student's t-test, *p<0.05. (B) Recruitment of neutrophils to the lungs of W W and W K mice during infection with M. tuberculosis. Neutrophils were quantitated by flow cytometry using single cell suspensions stained with anti-CD11b and anti-Gr1 antibodies. Results are expressed as the average number (±S.E.) of CD11bhiGr1hi cells per lung and for 4 mice per time point and per group. Data were compared using a two-tailed Student's t-test, *p<0.05, **p<0.01, ***p<0.005. (C) Representative dot plots showing the proportion of CD11bhiGr1hi cells in W W and W K mice 14 weeks post-infection with M. tuberculosis.

FIG. 7. Flow cytometry analysis of lymphocytes (A) and myeloid cells (B) in the lungs of M. tuberculosis infected chimeric mice. Single cell suspensions were stained using anti-CD4, anti-CD8, anti-CD11b, anti-CD11c and anti-Gr1 antibodies. Results are expressed as the average number (±S.E.) of CD4+ and CD8+ cells (A), CD11chiCD11blo (alveolar macrophages), CD11c-CD11bhiGr-1− (monocytes), CD11cloCD11bhi (interstitial macrophages) and CD11chiCD11bhi (myeloid dendritic cells) cells (B) per lung and for 4 mice per time point and per group. Data were compared using a two-tailed Student's t-test. *p<0.1.

FIG. 8. Differential Expression of IFNγ-Responsive Genes in the Lungs of Chimeric Mice Infected with M. tuberculosis for 9 Weeks.

Microarray analysis was conducted on pools of RNA isolated from 4 mice per group and the pools of each group were hybridized against each other. The results are expressed as the fold change in mRNA expression in W K mice over W W mice. Dotted lines represent a two-fold change in expression.

FIG. 9. IFNγ-Dependent Expression of IDO in Nonhematopoietic Cells is Impaired in the Lungs of W K Mice Infected with M. tuberculosis.

(A-B) Quantitative real-time PCR (qRT-PCR) evaluation of Ido (A) and Ifng (B) mRNA expression in the lungs of chimeric mice after infection with M. tuberculosis. Results are expressed as the average relative level of expression (±S.E.) of specific mRNA after normalization to 18S ribosomal RNA for 4 mice per time point and per group. Data were compared using a two-tailed Student's t-test, *p<0.05, **p<0.01. (C) Expression of IDO in lung sections of chimeric mice 15 weeks after infection with M. tuberculosis. Positive immunohistochemical staining in airway epithelial (arrows), vascular endothelial (white arrowheads) and myeloid (black arrowheads) cells. Original magnification, x400.

FIG. 10 Induction of Idol expression by IFNγ in NIH/3T3 murine fibroblasts, measured by quantitative real-time (qRT) PCR. Cells were seeded in 6-well plates at a density of 106 cells/well, in DMEM supplemented with 10% heat-inactivated FCS. They were treated with 20 ng/ml of recombinant murine IFN in triplicate and cultured for 24 h at 37° C. under 5% CO2 atmosphere. Control cells were maintained in culture media alone. Total RNA was extracted and purified using QIA shredder columns and RNeasy Mini Kit (Qiagen). After DNAse treatement (Ambion), 1 μg of RNA was retro-transcribed into cDNA and used as template in a qRT-PCR reaction using primers specific for Ido. Results are expressed as the average relative level of expression (±S.E.) of specific mRNA after normalization to GAPDH expression. Data for control and IFN-treated cells were compared using a two-tailed Student's t-test.

FIG. 11. Il17a Expression During M. tuberculosis Infection In Vivo and its Regulation by Kynurenines In Vitro.

(A) qRT-PCR evaluation of Il17a, Il23a, Tgfb1 and Il16 mRNA expression in the lungs of chimeric mice after infection with M. tuberculosis. Results are expressed as the average relative level of expression (±S.E.) of specific mRNA after normalization to 18S ribosomal RNA for 4 mice per time point and per group. IL17a and Il23a mRNA expression was quantitated 3, 4, 9, and 14 weeks post-infection, Tgfb1 and Il6 mRNA expression was assayed on samples harvested on week 9 post-infection. Data were compared using a two-tailed Student's t-test, *p<0.05, **p<0.01 or indicated value. (B) Dose response of IL-17 production by differentiating Th17 cells in vitro in the presence of increasing concentrations of tryptophan catabolites (L-kynurenine, 3′-hydroxy-DL-kynurenine, 3′-hydroxyanthranilic acid, anthranilic acid and quinolinic acid). A nonlinear regression with variable slope analysis was applied using Prism software (GraphPad) to determine an IC50 value of 11.7±1.1 (C) IL-17 production by differentiating Th17 cells in vitro in the presence of 15 μM of tryptophan catabolites after 6 days of culture, in the absence of IL-23 (white bars), in the presence of IL-23 for the last 3 days (grey bars) or for 6 days of culture (black bars). Each condition was assayed in triplicate. The results are expressed as the mean concentration (±S.E.) of IL-17 in the culture supernatants after 6 days of incubation as measured by ELISA and are representative of two independent experiments.

FIG. 12. Mycobacteriostatic activity of tryptophan catabolites was measured by adding increasing concentrations, from 16 to 4096 μg/ml, of an equiweight mixture of L-kynurenine, 3-hydroxy-DL-kynurenine, anthranilic acid, 3-hydroxyanthranilic acid and quinolinic acid to a liquid culture of Mycobacterium tuberculosis strain H37Rv in 7H9-Tween 0.05% broth, enriched with ADC. The cultures were incubated at 37° C. under agitation for 4 days. The bacterial growth was monitored every day by measuring the optical density of the cultures at 580 nm (OD580 nm). The cultures were realized in triplicates for each concentration and the results are expressed as the average OD580 nm (±S.E.) per concentration and per time point. The concentration of tryptophan catabolites inhibiting 50% of M. tuberculosis growth (IC50) was calculated using a nonlinear regression with variable slope analysis (Prism software—GraphPad). At mid-log phase (day 3 of culture), the IC50 was 240.6±1.1 μg/ml. As a control, Escherichia coli (ATCC11775) was grown in LB broth at 37° C. under agitation in the presence of increasing concentrations of the tryptophan catabolites mixture. The OD of the cultures was measured at 600 nm every hour for 4 hours. The IC50 determined at mid-log phase was 398.6±1.0 μg/ml (data not shown).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

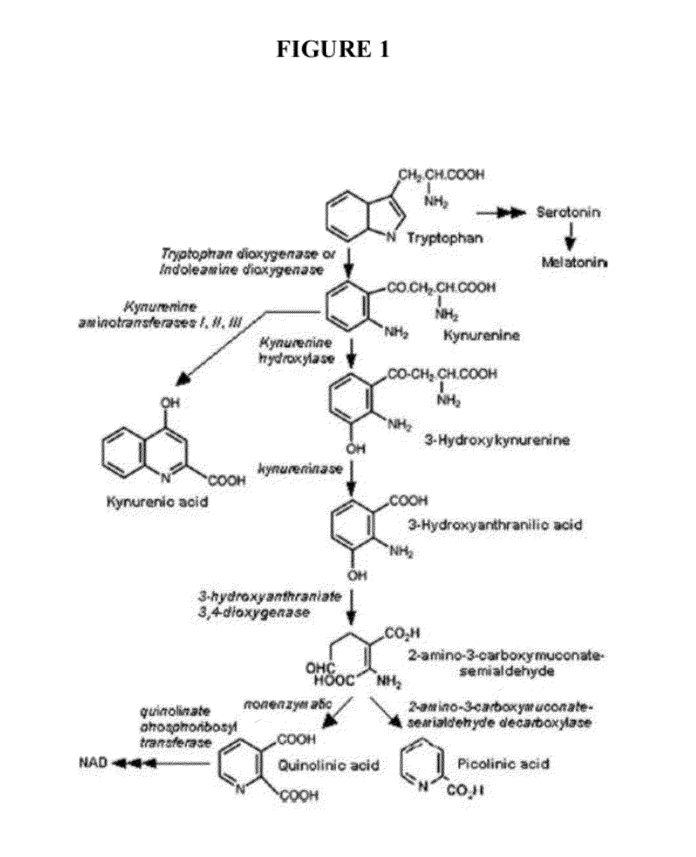

The terms “tryptophan metabolites”, “kynurenine”, “kynurenines”, kynurenine analogs”, and any variants not specifically listed, may be used herein interchangeably, and as used throughout the present application and claims refer to compounds, particularly structures and endogenous or non endogenous compounds, and extends to those having the structures and chemical natures described herein and presented in FIG. 1, and the profile of activities set forth herein and in the Claims. Accordingly, compounds or agents displaying substantially equivalent or altered activity are likewise contemplated. These modifications may be deliberate, for example, such as modifications obtained through alteration or addition of specific chemical groups, site-directed mutagenesis, or may be accidental, such as those obtained through mutations in hosts that are producers of the complex or its named subunits. Also, the terms “tryptophan metabolites”, “kynurenine”, “kynurenines”, kynurenine analogs” are intended to include within their scope structures and compounds specifically recited herein, including DL-kynurenine, L-kynurenine, 3-hydroxy-DL-kynurenine and 3-hydroxy-anthranilic acid, as well as all substantially homologous analogs and variations.

The amino acid residues and amino acid derivative structures described herein are preferred to be in the “L” isomeric form. However, residues in the “D” isomeric form or in the combined isomer DL form, may be tested and potentially substituted for any L-amino acid residue, provided that the desired functional property of Th17 cell modulation is retained by the polypeptide.

NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

The phrase “pharmaceutically acceptable” refers to molecular entities and compositions that are physiologically tolerable and do not typically produce an allergic or similar untoward reaction, such as gastric upset, dizziness and the like, when administered to a human.

‘Therapeutically effective amount’ means that amount of a drug, compound, expression inhibitory agent, or pharmaceutical agent that will elicit the biological or medical response of a subject that is being sought by a medical doctor or other clinician. Thus, the phrase “therapeutically effective amount” is used herein to mean an amount sufficient to prevent, and preferably reduce by at least about 30 percent, more preferably by at least 50 percent, most preferably by at least 90 percent, a clinically significant change in a target cell (e.g. cell division, proliferation, activity) or target cellular mass, or other feature of pathology as may attend its presence and activity.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense.

As used herein, the term ‘antagonist’ is used to describe a compound that does not provoke a biological response itself upon binding to a receptor, but blocks or dampens agonist-mediated responses.

The term ‘assay’ means any process used to measure a specific property of a compound. A ‘screening assay’ means a process used to characterize or select compounds based upon their activity from a collection of compounds.

The term ‘carrier’ means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term ‘compound’ may be used herein in the context of a ‘test compound’ or a ‘drug candidate compound’ described in connection with the assays of the present invention. As such, these compounds comprise organic or inorganic compounds, derived synthetically, recombinantly, or from natural sources. The compounds may include inorganic or organic compounds such as polynucleotides, lipids or hormone analogs. Other biopolymeric organic test compounds include peptides comprising from about 2 to about 40 amino acids and larger polypeptides comprising from about 40 to about 500 amino acids, including polypeptide ligands, enzymes, receptors, channels, antibodies or antibody conjugates.

The term ‘condition’ or ‘disease’ means the overt presentation of symptoms (i.e., illness) or the manifestation of abnormal clinical indicators (for example, biochemical indicators or diagnostic indicators). Alternatively, the term ‘disease’ refers to a genetic or environmental risk of or propensity for developing such symptoms or abnormal clinical indicators.

The term ‘contact’ or ‘contacting’ means bringing at least two moieties together, whether in an in vitro system or an in vivo system.

The term ‘inhibit’ or ‘inhibiting’, in relationship to the term ‘response’ means that a response is decreased or prevented in the presence of a compound as opposed to in the absence of the compound.

The term ‘inhibition’ refers to the reduction, down regulation of a process or the elimination of a stimulus for a process, which results in the absence or minimization of the expression or activity of a cell, a protein or polypeptide.

The term ‘induction’ refers to the inducing, up-regulation, or stimulation of a process, which results in the expression or activity of a cell, a protein or polypeptide.

The term ‘ligand’ means an endogenous, naturally occurring molecule specific for an endogenous, naturally occurring receptor.

The term ‘pharmaceutically acceptable salts’ refers to the non-toxic, inorganic and organic acid addition salts, and base addition salts, of compounds as disclosed herein. These salts can be prepared in situ during the final isolation and purification of compounds useful in the present invention.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine in advance of a visit to a geographical region where malaria is endemic or the risk of contracting malaria is high.

The term ‘solvate’ means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term ‘subject’ includes humans and other mammals.

The term ‘treating’ or ‘treatment’ of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, ‘treating’ or ‘treatment’ relates to slowing the progression of the disease.

The term “disease characterized by inflammation” or “inflammatory disease” or “inflammatory condition” refers to a disease which involves, results at least in part from or includes inflammation. The term includes, but is not limited to, exemplary diseases selected from allergic airways disease (e.g. asthma, rhinitis), autoimmune diseases, transplant rejection, Crohn's disease, rheumatoid arthritis, psoriasis, juvenile idiopathic arthritis, colitis, and inflammatory bowel diseases.

The term “autoimmune disease” refers to a disease which involves, results at least in part from or includes an immune response of the body against substances and tissues normally present in the body. The term includes, but is not limited to, exemplary diseases selected from Addison's disease, ankylosing spondylitis, coeliac disease, chronic obstructive pulmonary disease, dermatomyositis, diabetes mellitus type 1, Graves' disease, Guillain-Barré syndrome (GBS), lupus erythematosus, multiple sclerosis, myasthenia gravis, rheumatoid arthritis, and vasculitis.

The term “cancer” refers to a malignant or benign growth of cells in skin or in body organs, for example but without limitation, breast, prostate, lung, kidney, pancreas, stomach or bowel. A cancer tends to infiltrate into adjacent tissue and spread (metastasize) to distant organs, for example to bone, liver, lung or the brain. As used herein the term cancer includes both metastatic rumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma.

As used herein, “pg” means picogram, “ng” means nanogram, “ug” or “μg” mean microgram, “mg” means milligram, “ul” or “μl” mean microliter, “ml” means milliliter, “l” means liter.

A “promoter sequence” is a DNA regulatory region capable of binding RNA polymerase in a cell and initiating transcription of a downstream (3′ direction) coding sequence. The promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used or applicable herein, is defined as a molecule comprised of two or more ribonucleotides, preferably more than three. Its exact size will depend upon many factors which, in turn, depend upon the ultimate function and use of the oligonucleotide.

The term “primer” refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₙ, with washes of higher stringency, if desired.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Amino acid substitutions may also be introduced to substitute an amino acid with a particularly preferable property. For example, a Cys may be introduced a potential site for disulfide bridges with another Cys. A H is may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein. Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

Th17 cells play a role in inflammation and tissue injury in inflammatory and immune system diseases. Th17 cells have been broadly implicated in autoimmune disease and Th17 cells have been shown to be highly pathogenic. Other studies of Th17 cells have demonstrated preferential induction of IL-17 in cases of host infection with various bacterial and fungal species. The present studies and examples demonstrate that IFNγ-responsive cells, and particularly including IFNγ-responsive nonhematopoietic cells, are required for control of M. tuberculosis infection. In the absence of IFNγR on nonhematopoietic cells, mice succumb to M. tuberculosis infection, with severe inflammation in the lungs. In particular, IFNγR-deficient nonhematopoietic cells under-express indoleamine-2,3-dioxygenase (IDO) in lung epithelium and endothelium during chronic tuberculosis, and this was accompanied by over-expression of IL-17 and massive recruitment of neutrophils to the lungs. IL-17 overexpression is mediated directly by Th17 cells, particularly unchecked Th17 responses. Individualkynurenines have now been shown to have a direct and additive inhibitory effect on Th17 cells. The invention thus contemplates the use and application of kynurenines or kynurenine analogs or antagonists in modulating Th17 cell function, differentiation and/or activity, and in modulating IL-17 production in immune cell responses and inflammation. Further, the action of kynurenines is mediated via IL-23, particularly by inhibition of the effects of IL-23.

Tryptophan derivatives have been identified as naturally occurring ligands of arylhydrocarbon receptor (Ahr), a basic-helix-loop-helix transcription factor. Ahr is highly expressed in Th17 cells and plays a role in promoting the differentiation of Th17 cells and in inducing them to secrete cytokines (IL-22) (Schmidt, J V et al (1996) PNAS 93:6731-6736; Veldpen, M et al (2008) Nature 453:106-109; Quintana, F J et al (2008) Nature 453:65-71). Ahr knockout animals have been utilized to study Ahr's physiological role in detoxification and in the immune system (Fernandex-Salguero, P M et al (1997) Vet Pathol 34:605-614; Esser, C. (2009) Biochem Pharmacol 77:597-607). Studies of the action of tryptophan metabolites and kynurenines on Ahr and their modulation of Th17 cells via Ahr and in Ahr defective animals will further elucidate the mechanisms of tryptophan metabolite and kynurenine modulation of Th17 cells and immune response.

The present invention relates generally to the modulation of T cells, and particularly to the modulation of IL-17 expressing cells. The present invention relates generally to the modulation of T cells, and particularly Th17 cells and their associated activities or cytokines, particularly IL-17 and IL-23. The present invention describes that tryptophan metabolites, kynurenines, provided as a mixture, or as individual compounds, specifically modulate IL-17 expressing cells, Th17 cells. Tryptophan metabolites, kynurenines, provided as a mixture, or as individual compounds, inhibit differentiation of naive T lymphocytes to Th17 effector cells, with effects on Th1 differentiation only at higher (i.e., 5-fold) concentrations. The invention provides kynurenines, particularly L-kynurenine, 3-hydroxy-DL-kynurenine, and 3-hydroxyanthranilic acid, as modulators of Th17 cells and their activity, including for the inhibition of IL-17 production and the inhibition of IL-23 action.

Modulation of T lymphocyte differentiation, particularly of IL-17 expressing cells, particularly of Th17 cells, has multiple potential applications, including for treatment of inflammatory diseases, including autoimmune diseases and inflammation associated with acute and chronic infectious diseases. In addition, modulation of T lymphocyte differentiation in conjunction with vaccination has application in enhancing the efficacy of certain vaccines, and may prevent adverse effects of certain vaccines. Inhibition of Th17 cell activation or activity has application in reducing or alleviating inflammation, inflammatory conditions and auto-immune disorders or conditions. Stimulation of Th17 cells has implications in and potential application for cancer immunity, cancer therapy, and antimicrobial immunity and clearance of microbes or fungi.

As lead compounds, kynurenines or kynurenine analogs provide anti-inflammatory and/or immunomodulatory drugs or compounds. In addition, they have use and application in further characterizing the molecular mechanisms of inhibition of Th17 differentiation and in the discovery, screening and development of analogs with enhanced potency and efficacy in treatment and modulation of disease and the immune response.

Kynurenine antagonists or inhibitors of tryptophan metabolic pathway enzymes, such as IDO, may be utilized in stimulating or activating Th17 cells or in induction of IL-17 expression. Activation or stimulation of Th17 cells has uses and application in cancer immunity, cancer therapy and microbial immunity and clearance.

The kynurenines or agents exhibiting either mimicry or antagonism to them or control over their production or generation, may be prepared in pharmaceutical compositions, with a suitable carrier and at a strength effective for administration by various means to a patient experiencing an adverse medical condition associated with specific Th17 cell activity or cell factors for the treatment or alleviation thereof. A variety of administrative techniques may be utilized, among them parenteral techniques such as subcutaneous, intravenous and intraperitoneal injections, catheterizations and the like. Average quantities of the kynurenines or their analogs may vary and in particular should be based upon the recommendations and prescription of a qualified physician or veterinarian.

Also, antibodies including both polyclonal and monoclonal antibodies, and drugs that modulate the production or activity of the kynurenines and/or their metabolites may possess certain diagnostic applications and may for example, be utilized for the purpose of detecting and/or measuring conditions such as inflammation, immune system response, or the like. For example, the kynurenines, IDO enzyme, IL-17, IL-23 may be used to produce both polyclonal and monoclonal antibodies to themselves in a variety of cellular media, by known techniques (such as the hybridoma technique utilizing, for example, fused mouse spleen lymphocytes and myeloma cells). Likewise, small molecules that mimic or antagonize the activity(ies) of the kynurenines of the invention may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced can be screened for various properties; i.e., isotype, epitope, affinity, etc. Of particular interest are monoclonal antibodies that neutralize the activity of IL-17 or of Th17 cells. Such monoclonals can be readily identified in Th17 cell or IL-17 activity assays. High affinity antibodies are also useful when immunoaffinity purification of Th17 cells, native or recombinant proteins is desired or possible. In addition, it may be preferable for the antibody molecules used herein be in the form of Fab, Fab′, F(ab′)₂ or F(v) portions of whole antibody molecules.

The present invention contemplates therapeutic compositions useful in practicing the therapeutic methods of this invention. A subject therapeutic composition includes, in admixture, a pharmaceutically acceptable excipient (carrier) and one or more of a kynurenine, kynurenine analog, tryptophan metabolite, analog thereof, or antagonist thereof, as described herein as an active ingredient. In a preferred embodiment, the composition comprises one or more of a kynurenine capable of modulating a target Th17 cell and/or capable of altering IL-17 production by cells including such cells.

The preparation of therapeutic compositions which contain compounds, amino acid analogs, analogs, agonists or antagonists as active ingredients is well understood in the art. Typically, such compositions are prepared as oral formulations or as injectables, either in tablet form or as liquid solutions or suspensions, and also solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

One or more kynurenine, analog or antagonist can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic kynuenine-, analog- or antagonist-containing compositions are conventionally administered orally or parenterally, as by ingestion or injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of modulation, inhibition or activation of IL-17 expressing cells, particularly of Th17 cells or Th17 cell-related activity or expression desired. Precise amounts of active ingredient required to be administered depend on the judgment of the practitioner and are peculiar to each individual. However, suitable dosages will be based on a physiologically or clinically relevant amount of active ingredient per kilogram body weight of individual per day and depend on the route of administration. Suitable regimes for initial administration and further or continued administration or shots are also variable, but are typified by an initial administration followed by repeated doses at one or more hour intervals by a subsequent injection or other administration. Alternatively, continuous intravenous infusion sufficient to maintain relevant concentrations in the blood are contemplated.

The therapeutic compositions may further include an effective amount of the tryptophan metabolite, kynurenine, antagonist or analog thereof, and one or more of the following active ingredients: a cytokine, an immune modulator, an antibiotic, a steroid.

The present invention also provides biologically compatible compositions which can act to modulate, including inhibit, Th17 cells wherein said compositions comprise an effective amount of one or more compounds identified as kynurenine or tryptophan metabolite analogs or antagonists, and/or the kynureninee as described herein. The present invention also provides biologically compatible compositions which can act to modulate, including inhibit, IL-17 expressing cells wherein said compositions comprise an effective amount of one or more compounds identified as kynurenine or tryptophan metabolite analogs or antagonists, and/or the kynureninee as described herein.

A biologically compatible composition is a composition, that may be solid, liquid, gel, or other form, in which the one or more kynurenine, compound, agent of the invention is maintained in an active form, e.g., in a form able to effect a biological activity. For example, a compound of the invention would have modulatory activity on Th17 cells, or on IL-17 production, or inhibit IL-23, or demonstrate anti-inflammatory activity, etc. as described herein.

A particular biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well-known non-toxic physiologically acceptable carriers, adjuvants and vehicles as desired.

A particular embodiment of the present composition invention is a pharmaceutical composition comprising a therapeutically effective amount of one or more kynurenine, tryptophan metabolite, analog or antagonist thereof, as described hereinabove, in admixture with a pharmaceutically acceptable carrier. Another particular embodiment is a pharmaceutical composition for the treatment or prevention of a disease characterized by Th17 cell activity, including excessive or reduced Th17 cells, such as inflammation, allergic and autoimmune diseases, and cancer, or a susceptibility to said disease, comprising an effective amount of one or more kynurenine, tryptophan metabolite, analog or antagonist thereof, its pharmaceutically acceptable salts, hydrates, solvates, or prodrugs thereof in admixture with a pharmaceutically acceptable carrier. A further particular embodiment is a pharmaceutical composition for the treatment or prevention of a disease involving inflammation, or a susceptibility to the condition, comprising an effective amount of the one or more kynurenine, tryptophan metabolite, analog or antagonist thereof, its pharmaceutically acceptable salts, hydrates, solvates, or prodrugs thereof in admixture with a pharmaceutically acceptable carrier. A further particular embodiment is a pharmaceutical composition for the treatment or prevention of an autoimmune disease, or a susceptibility to said disease, comprising an effective amount of the one or more kynurenine, tryptophan metabolite, analog or antagonist thereof, its pharmaceutically acceptable salts, hydrates, solvates, or prodrugs thereof in admixture with a pharmaceutically acceptable carrier.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The agents or compositions of the invention may be combined for administration with or embedded in polymeric carrier(s), biodegradable or biomimetic matrices or in a scaffold. The carrier, matrix or scaffold may be of any material that will allow composition to be incorporated and expressed and will be compatible with the addition of cells or in the presence of cells. Particularly, the carrier matrix or scaffold is predominantly non-immunogenic and is biodegradable. Examples of biodegradable materials include, but are not limited to, polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to administration or implantation, e.g., by treatment with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof. Matrices suitable include a polymeric mesh or sponge and a polymeric hydrogel. In the particular embodiment, the matrix is biodegradable over a time period of less than a year, more particularly less than six months, most particularly over two to ten weeks. The polymer composition, as well as method of manufacture, can be used to determine the rate of degradation. For example, mixing increasing amounts of polylactic acid with polyglycolic acid decreases the degradation time. Meshes of polyglycolic acid that can be used can be obtained commercially, for instance, from surgical supply companies (e.g., Ethicon, N.J.). In general, these polymers are at least partially soluble in aqueous solutions, such as water, buffered salt solutions, or aqueous alcohol solutions that have charged side groups, or a monovalent ionic salt thereof.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

The active one or more kynurenine, tryptophan metabolite, analog or antagonist thereof may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

As defined above, therapeutically effective dose means that amount of one or more kynurenine, tryptophan metabolite, analog or antagonist thereof, which alter Th17 rcell responses or activity, alter IL-17 production, or ameliorate one or more of the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, and it can be expressed as the ratio, LD₅₀/ED₅₀. Pharmaceutical compositions that exhibit large therapeutic indices are preferred. The data obtained from cell culture assays and animal studies are used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by direct injection, transdermal absorption, catheter, infusion pump or stent. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al WO 94/02595.

It is further intended that kynurenine analogs may be prepared and derived from the tryptophan metabolites' chemical structures within the scope of the present invention. Analogs exhibiting “IL-17 expression modulating activity”, “Th17 modulating activity” or “kynurenine activity” such as small molecules, chemical compounds, mimics, etc, whether functioning as promoters or inhibitors, may be identified by known in vivo and/or in vitro assays, or assays developed by one skilled in the art.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

In accordance with the above, an assay system for screening potential drugs effective to mimic or inhibit physiologicalkynurenines and/or modulate the activity of IL-17 expressing cells may be prepared. In accordance with the above, an assay system for screening potential drugs effective to mimic or inhibit physiologicalkynurenines and/or modulate the activity of Th17 cells may be prepared. Th17 cells, or other IL-17 expressing cells or cell compositions, or one or more kynurenine may be introduced into a test system, and the prospective drug may also be introduced into the resulting cell culture, and the culture thereafter examined to observe any changes in the activity of the cells or in the amounts of interleukins or factors such as IL-17, due either to the addition of the prospective drug alone, or due to the effect of added quantities of the known kynurenine or Th17 cells.

In an additional aspect, the present invention relates to a method for assaying for drug candidate compounds that modulate Th17 cells, comprising contacting the compound with Th17 cells under conditions that allow said cells to bind to or otherwise associate with the compound, and detecting a change in the activity or amount of Th17 cells. In particular, said method may be used to identify drug candidate compounds able to suppress the release of cytokines from Th17 cells.

Therefore, in one aspect, the present invention relates to a method for assaying for drug candidate compounds that modulate Th17 cells comprising contacting the compound with Th17 cells or a cellular sample including Th17 cells, in the presence or absence of one or more kynurenine, under conditions that allow said compound to act on or come in contact with the cells, and detecting the activity of the Th17 cells or of the kynurenine. In particular said method may be used to identify drug candidate compounds that modulate the release of cytokines from Th17 cells, in particular IL-17.

In another aspect, the present invention relates to a method for assaying for drug candidate compounds that modulate IL-17 expressing cells comprising contacting the compound with IL-17 expressing cells or a cellular sample including IL-17 expressing cells, in the presence or absence of one or more kynurenine, under conditions that allow said compound to act on or come in contact with the cells, and detecting the activity of the IL-17 expressing cells or of the kynurenine.

In a further such aspect, the present invention relates to a method for assaying for drug candidate compounds that inhibit Th17 cells comprising contacting the compound with Th17 cells or a cellular sample including Th17 cells, in the presence or absence of one or more kynurenine, under conditions that allow said compound to act on or come in contact with the cells, and detecting the activity of the Th17 cells or of the kynurenine. In particular said method may be used to identify drug candidate compounds that inhibit the release of cytokines from Th17 cells, in particular IL-17. In particular said method may be used to identify drug candidate compounds that inhibit the release of cytokines from IL-17 expressing cells.

More particularly, the invention relates to a method for identifying an agent or compound that modulates Th17 cells said method comprising:

(a) contacting a population of mammalian cells including Th17 cells with one or more compound that exhibits kynurenine activity or a candidate compound to determine its kynurenine-like activity, and
(b) measuring a property related to Th17 cell activity, differentiation, or proliferation.

In a further aspect of the present invention said method is used to identify a compound that alters the release of cytokines from Th17 cells. In particular the release of IL-17 is measured. In a further aspect of the present invention said method is used to identify a compound that alters the release of cytokines from IL-17 expressing cells.

The invention relates to a method for identifying an agent or compound that inhibits IL-17 expressing cells said method comprising:
(a) contacting a population of mammalian cells including IL-17 expressing cells with one or more compound that exhibits kynurenine activity or a candidate compound to determine its kynurenine-like activity, and
(b) measuring a property related to IL-17 expressing cell activity, differentiation, or proliferation so as to determine its reduction or inhibition.

The invention relates to a method for identifying an agent or compound that inhibits Th17 cells said method comprising:
(a) contacting a population of mammalian cells including Th17 cells with one or more compound that exhibits kynurenine activity or a candidate compound to determine its kynurenine-like activity, and
(b) measuring a property related to Th17 cell activity, differentiation, or proliferation so as to determine its reduction or inhibition.

In a further aspect of the present invention said method is used to identify a compound that inhibits the release of cytokines from Th17 cells. In particular the release of IL-17 is measured.

In particular said method may be used to identify drug candidate compounds capable of suppressing the release of cytokines from Th17 cells. One particular means of measuring the activity or expression of the cytokine polypeptide(s) is to determine the amount of said polypeptide using a polypeptide binding agent, such as an antibody, or to determine the activity of said polypeptide in a biological or biochemical measure, for instance the amount of phosphorylation of a target of a kinase polypeptide.

Depending on the choice of the skilled artisan, the present assay method may be designed to function as a series of measurements, each of which is designed to determine whether the drug candidate compound is indeed acting on the Th17 cells to thereby modulate Th17 cells. In addition, the present assay method may be designed to function as a series of measurements, one of which is designed to determine whether the drug candidate compound is indeed acting on Il-23 to thereby modulate Th17 cells. For example, an assay designed to determine the amount of cytokine, such as IL-17 or IL-23, may be necessary, but not sufficient, to ascertain whether the test compound would be useful for modulating Th17 cells directly when administered to a subject. Nonetheless, such information would be useful in identifying a set of test compounds for use in an assay that would measure a different property, for example further down the biochemical pathway, for example suppression of the release of cytokines from Th17 cells. Such additional assay(s) may be designed to confirm that the test compound, having kynurenine-like activity, actually modulates, for example inhibits, Th17 cells.

Suitable controls should always be in place to insure against false positive readings. In a particular embodiment of the present invention the screening method comprises the additional step of comparing the compound to a suitable control. In one embodiment, the control may be a cell or a sample that has not been in contact with the test compound. In an alternative embodiment, the control may be a cell that does not express the cytokine or a cellular sample that does not contain Th17 cells. Alternatively, in another aspect of such an embodiment, the cell in its native state does not express the cytokine and the test cell has been engineered so as to express the cytokine, so that in this embodiment, the control could be the untransformed native cell. Whilst exemplary controls are described herein, this should not be taken as limiting; it is within the scope of a person of skill in the art to select appropriate controls for the experimental conditions being used.

Analogous approaches based on art-recognized methods and assays may be applicable with respect to the compounds in any of various disease(s) characterized by Th17 cell activity, autoimmune response or inflammatory diseases. An assay or assays may be designed to confirm that the test compound, having kynurenine like, inhibits Th17 cells. In one such method the release of cytokines from Th17 cells is measured. In another such method the expression of cell surface markers on Th17 cells is measured.

The present assay method may be practiced in vitro or in vivo.

For high-throughput purposes, libraries of compounds may be used such as antibody fragment libraries, peptide phage display libraries, peptide libraries (e.g. LOPAP™, Sigma Aldrich), lipid libraries (BioMol), synthetic compound libraries (e.g. LOPAC™, Sigma Aldrich, BioFocus DPI) or natural compound libraries (Specs, TimTec).

Preferred drug candidate compounds are low molecular weight compounds. Low molecular weight compounds, i.e. with a molecular weight of 500 Dalton or less, are likely to have good absorption and permeation in biological systems and are consequently more likely to be successful drug candidates than compounds with a molecular weight above 500 Dalton (Lipinski et al. (1997)). Peptides comprise another class of drug candidate compounds. Peptides may be excellent drug candidates and there are multiple examples of commercially valuable peptides such as fertility hormones and platelet aggregation inhibitors. Natural compounds are another preferred class of drug candidate compound. Such compounds are found in and extracted from natural sources, and which may thereafter be synthesized. The lipids are another preferred class of drug candidate compound.

In vivo animal models of inflammation, inflammatory diseases, autoimmune disease or conditions, and T cell response and immunity models may be utilized by the skilled artisan to further or additionally screen, assess, and/or verify the agents or compounds identified in the present invention, including further assessing TARGET modulation in vivo. Such animal models include, but are not limited to, ulcerative colitis models, multiple sclerosis models (including EAE, lysolecithin-induced), arthritis models, allergic asthma models, airway inflammation models, and acute inflammation models.

A further aspect of the present invention relates to a method for modulating IL-17 expressing cells, comprising contacting said cell(s) with an agent or compound, including one or more kynurenine, tryptophan metabolite, or analog or antagonist thereof. Another aspect of the present invention relates to a method for modulating Th17 cells, comprising contacting said cell(s) with an agent or compound, including one or more kynurenine, tryptophan metabolite, or analog or antagonist thereof. In a further such aspect of the present invention a method is provided for inhibiting Th17 cells, comprising contacting said cell(s) with an agent or compound, including one or more kynurenine, tryptophan metabolite, or analog thereof.

In a further embodiment of this invention, commercial test kits suitable for use by a medical specialist may be prepared to determine the presence or absence of predetermined Th17 cell activity. In accordance with the testing techniques discussed above, one class of such kits will contain at least labeled Th17 cell cytokine, such as IL-17, or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for Th17 cell, comprising:
(a) a predetermined amount of at least one labeled immunochemically reactive component obtained by the direct or indirect attachment of a Th17 cell factor or a specific binding partner thereto, to a detectable label;
(b) other reagents; and
(c) directions for use of said kit.
   In a further variation, the test kit may be prepared and used for the purposes stated above, which operates according to a predetermined protocol (e.g. “competitive,” “sandwich,” “double antibody,” etc.), and comprises:
(a) a labeled component which has been obtained by coupling the factor or binding partner to a detectable label;
(b) one or more additional immunochemical reagents of which at least one reagent is a ligand or an immobilized ligand, which ligand is selected from the group consisting of:
   (i) a ligand capable of binding with the labeled component (a);
   (ii) a ligand capable of binding with a binding partner of the labeled component (a);
   (iii) a ligand capable of binding with at least one of the component(s) to be determined; and
   (iv) a ligand capable of binding with at least one of the binding partners of at least one of the component(s) to be determined; and
(c) directions for the performance of a protocol for the detection and/or determination of one or more components of an immunochemical reaction between the Th17 cell factor and a specific binding partner thereto.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The compound, factor or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### IFNγ-Responsive Nonhematopoietic Cells Regulate the Immune Response to Mycobacterium tuberculosis

Immunity to Mycobacterium tuberculosis in humans and in mice requires interferon gamma (IFNγ). While IFNγ has been studied extensively for its effects on macrophages in tuberculosis, we determined that protective immunity to tuberculosis also requires IFNγ-responsive nonhematopoietic cells. Bone marrow chimeric mice with IFNγ-unresponsive lung epithelial and endothelial cells exhibited earlier mortality and higher bacterial burdens than control mice, underexpressed indoleamine-2,3-dioxygenase (Ido) in lung endothelium and epithelium and overexpressed IL-17 with massive neutrophilic inflammation in the lungs. We also found that the products of IDO catabolism of tryptophan selectively inhibit IL-17 production by Th17 cells, by inhibiting the action of IL-23. These results reveal a previously-unsuspected role for IFNγ responsiveness in nonhematopoietic cells in regulation of immunity to M. tuberculosis, and reveal a novel mechanism for IDO inhibition of Th17 responses.

### Introduction

Interferon gamma (IFNγ) is essential to restrict progressive, fatal infection with Mycobacterium tuberculosis. Patients that are either deficient in IFNγ (Fieschi et al., 2003) or are incapable responding to IFNγ due to mutations in IFNγR1 or IFNγR2 suffer severe tuberculosis (Jouanguy et al., 1997), as well as infections with less virulent species of mycobacteria (Filipe-Santos et al., 2006). Likewise, IFNγ-deficient mice infected with M. tuberculosis die rapidly with high bacterial burdens in the lungs (Cooper et al., 1993; Flynn et al., 1993), implying that IFNγ contributes to the restriction of bacterial growth. Efforts to understand the actions of IFNγ that contribute to restriction and/or killing of M. tuberculosis have been confined to studies of macrophages, since these cells are known to harbor the bacteria in vivo, and since IFNγ was originally characterized as a macrophage-activating factor (Nathan, 1983). Subsequent studies have revealed essential roles for IFγ-responsive genes such as Nos2 (MacMicking et al., 1997) and Lrg-47 (MacMicking et al., 2003), which provide non-overlapping functions that restrict M. tuberculosis in cultured macrophages and in mice.

In addition, IFNγ treatment of cultured macrophages has been shown to promote acidification of mycobacterial phagosomes (Schaible et al., 1998) and autophagy, which can result in limited intracellular killing of M. tuberculosis (Gutierrez et al., 2004). While these studies have advanced our knowledge of the roles of IFNγ in the control of tuberculosis by immune cells, a comprehensive understanding of the contribution of IFNγ to immunity to M. tuberculosis is still lacking.

Since M. tuberculosis infection of cultured macrophages inhibits IFNγ induction of selected genes (Banaiee et al., 2006; Nagabhushanam et al., 2003; Ting et al., 1999), and since IFNγR and the molecules of the JAK/STAT pathway, required for IFNγ signaling, are expressed in diverse cell types, including nonhematopoietic cells such as epithelial and endothelial cells and fibroblasts (Schroder et al., 2004), we hypothesized that control of M. tuberculosis also requires responses to IFNγ by nonhematopoietic cells. To test this hypothesis, we used IFNγR1-deficient and wild type mice to prepare bone marrow chimeric mice whose hematopoietic and nonhematopoietic cells were selectively incapable of responding to IFNγ. We confirmed that early control of M. tuberculosis infection requires IFNγ-responsive hematopoietic cells; we found that long-term control of tuberculosis also requires IFNγ-responsive nonhematopoietic cells. In the absence of IFNγR on nonhematopoietic cells, mice succumb to M. tuberculosis infection, with severe inflammation in the lungs. Expression of IFNγ-responsive genes and immunohistochemical analyses revealed that IFNγR-deficient nonhematopoietic cells under-express indoleamine-2,3-dioxygenase in lung epithelium and endothelium during chronic tuberculosis, and this was accompanied by over-expression of IL-17 and massive recruitment of neutrophils to the lungs. These results extend the previously published observation that control of an intracellular pathogen requires IFNγ-responsive hematopoietic and nonhematopoietic cells when the pathogen invades both cell types (e.g., Toxoplasma gondii) (Yap and Sher, 1999). We have thus demonstrated that IFNγ-responsive nonhematopoietic cells are also required for control of M. tuberculosis, which, unlike T. gondii, resides predominantly, if not exclusively, in hematopoietic cells such as macrophages, dendritic cells, and neutrophils (Wolf et al., 2007).

### Results

Immune control of Tuberculosis Requires IFNγ-Responsive Nonhematopoietic Cells

To test the hypothesis that IFNγ contributes to immune control of tuberculosis by effects on cells other than macrophages, we examined the consequences of the absence of IFNγ responsiveness in hematopoietic versus nonhematopoietic cells on survival and pathology after a low-dose aerosol infection of mice with M. tuberculosis.

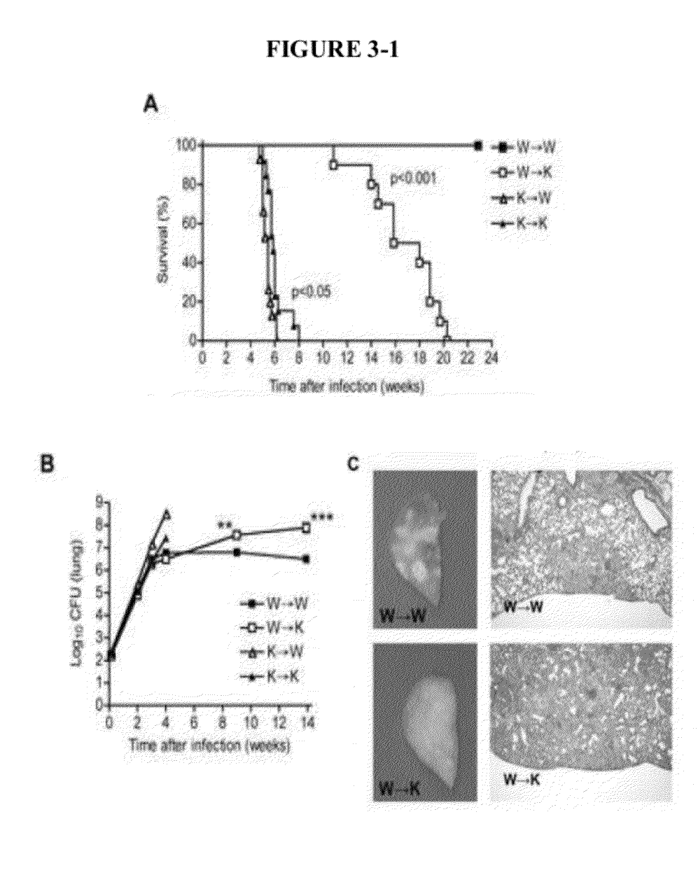

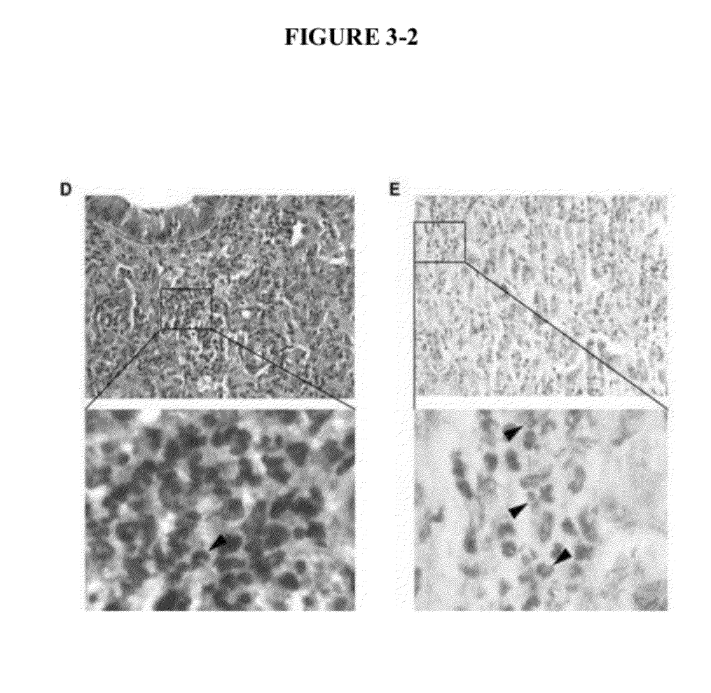

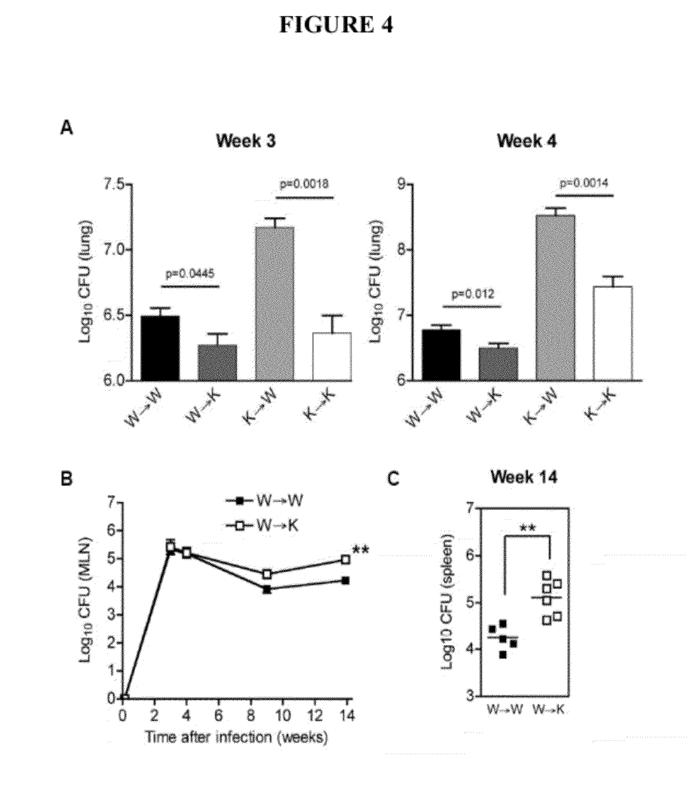

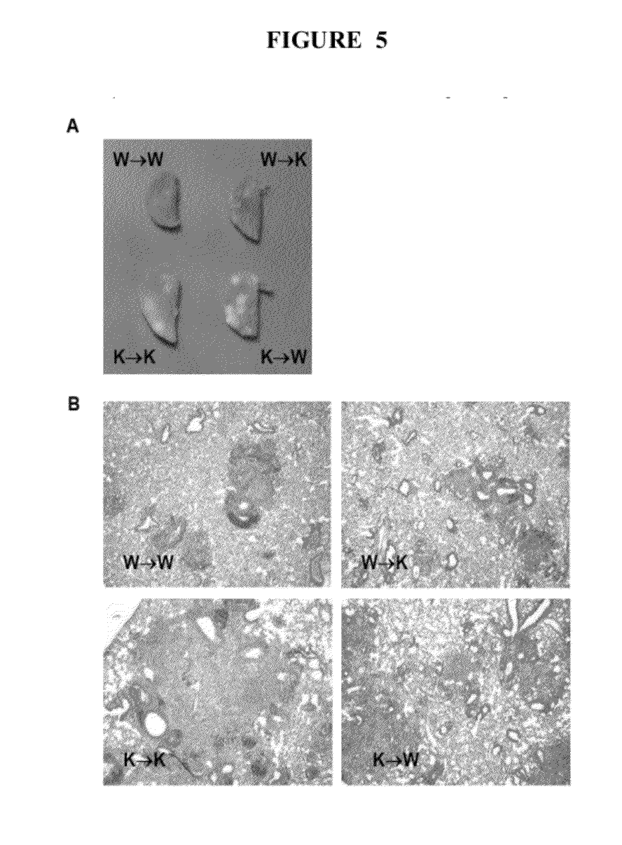

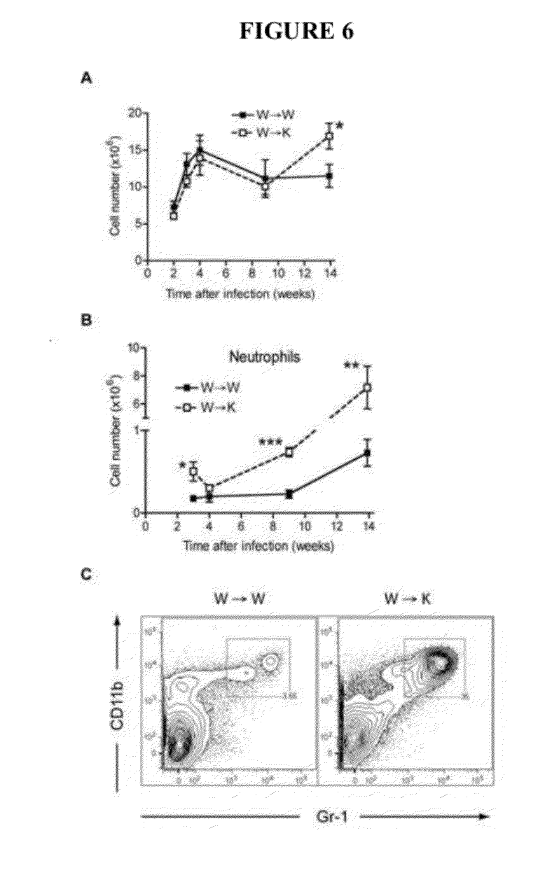

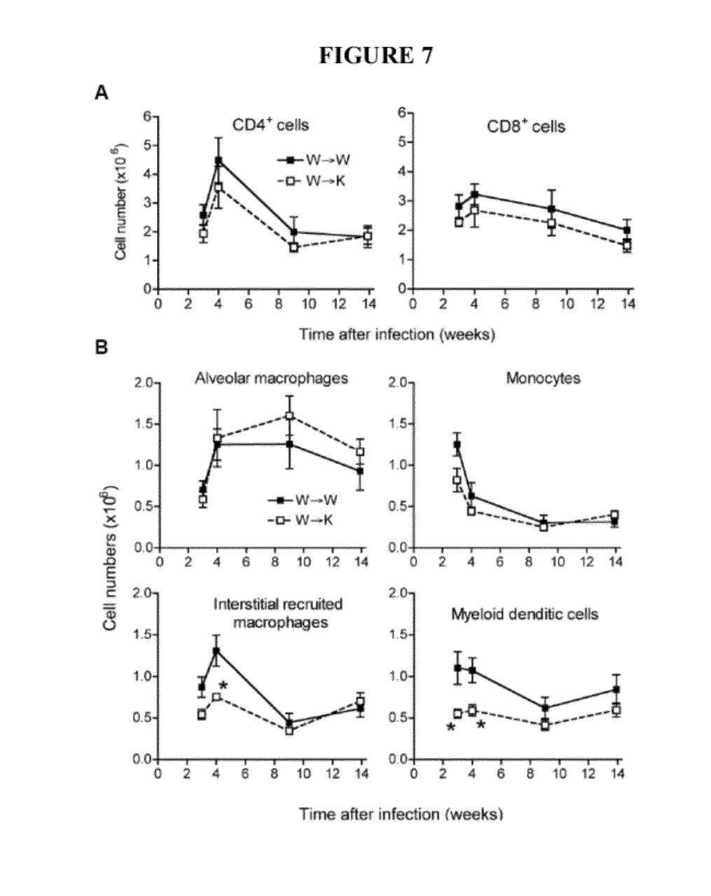

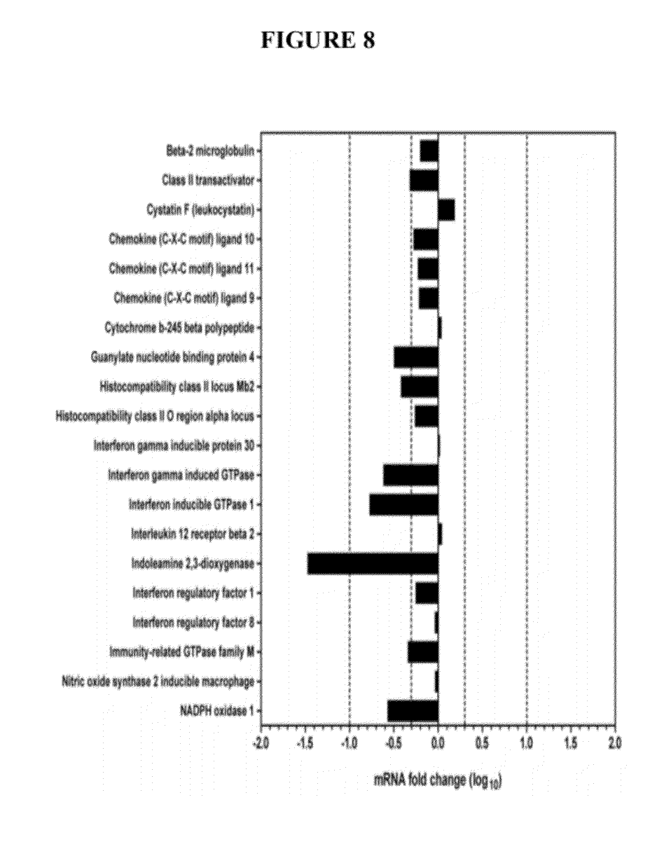

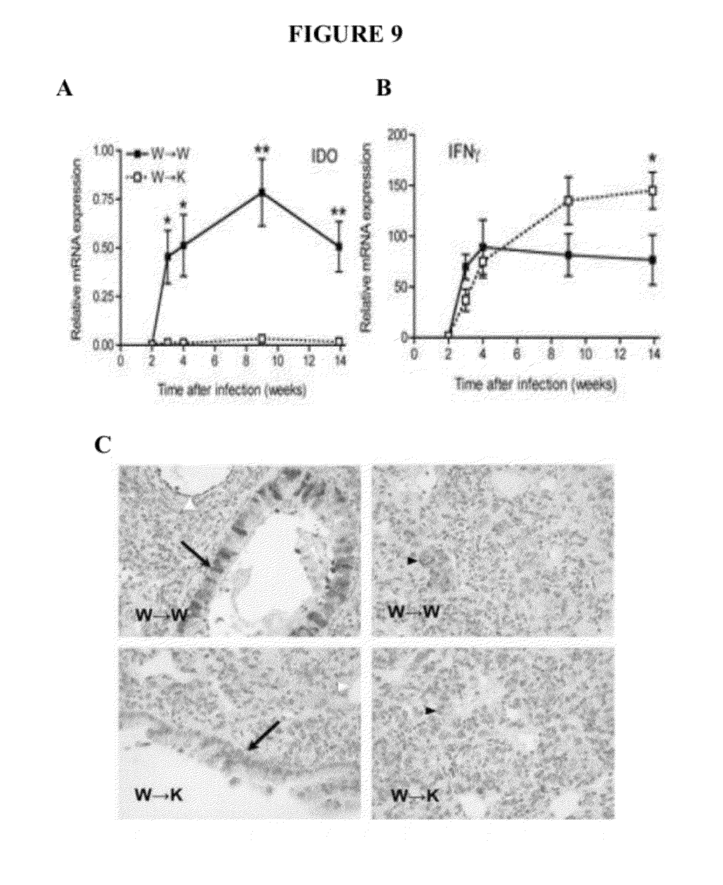

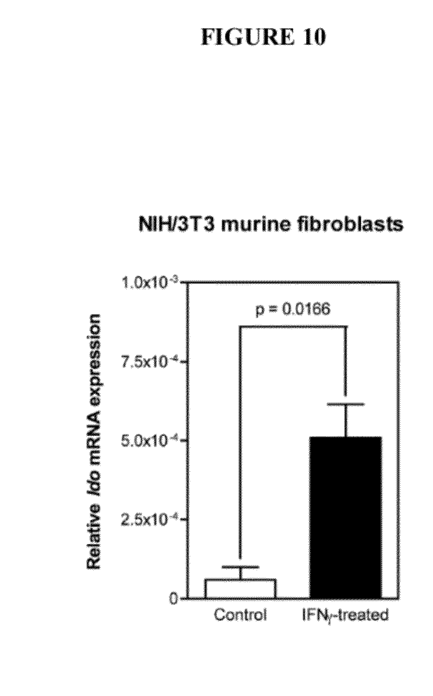

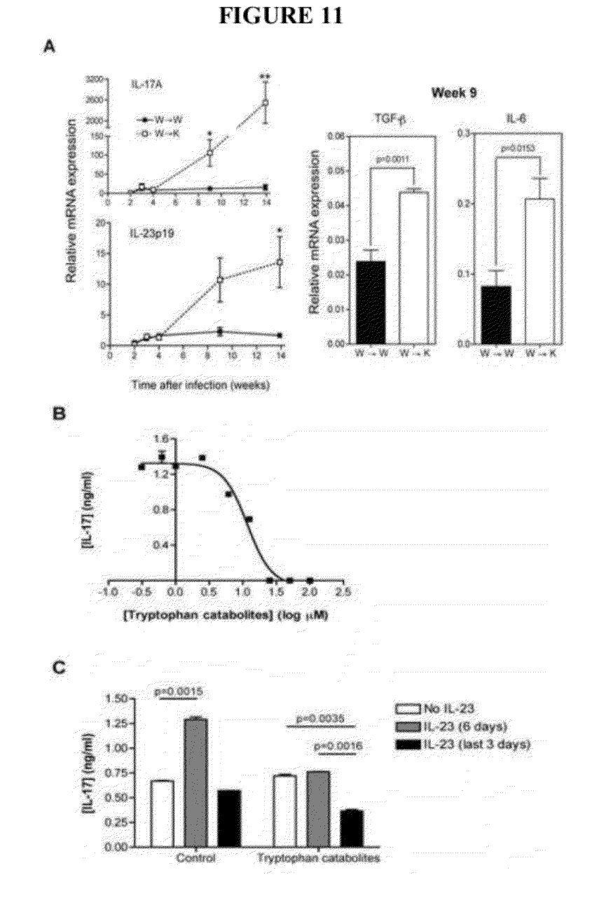

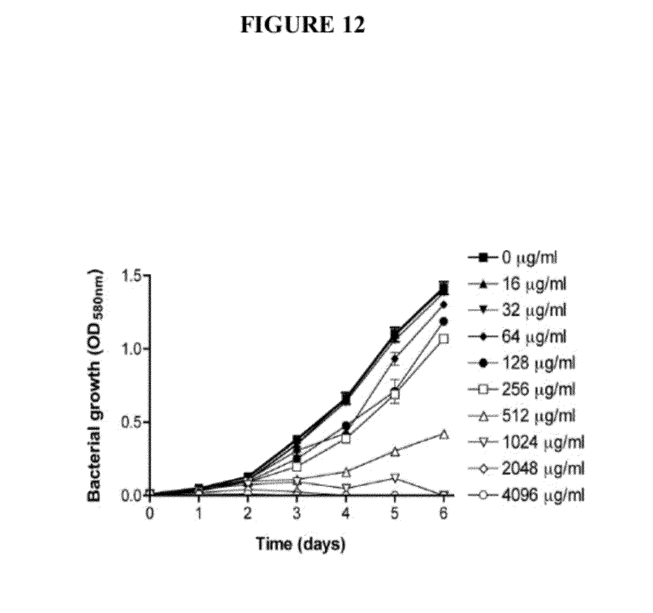

We found that chimeric mice reconstituted with IFNγR1−/− bone marrow cells died soon after infection, regardless of the presence of IFNγR on nonhematopoietic cells (FIG. 3A). Remarkably, wild type mice reconstituted with IFNγR1−/− bone marrow (K W mice) died significantly earlier than IFNγ R1−/− mice reconstituted with IFNγ R1−/− bone marrow (K K mice) (median survival=5.4±0.2 weeks vs. 5.9±0.3 weeks, p<0.05; FIG. 3A). The earlier death of K W mice was accompanied by higher bacterial burdens in the lungs than in K K mice as measured 3 weeks (6.4±0.1 vs. 7.2±0.1 log 10; p=0.0018; FIG. 3B and FIG. 4A) and 4 weeks post-infection (8.5±0.1 vs. 7.4±0.2 log 10; p=0.0014; FIG. 3B and FIG. 4A). The gross lung pathology of K W mice was also the most severe at that time (FIG. 5A) and, although the lesions appeared less extensive at the histological level than in K K mice, they were more multifocal (FIG. 5B). These results provide evidence that when hematopoietic cells, including macrophages, are incapable of responding to IFNγ, the response of nonhematopoietic cells to IFNγ is actually detrimental, as manifest by poorer control of bacterial growth in the lungs. Our observations in K K and K W mice also substantiate the long held belief that macrophages need to respond to IFNγ in order to control infection with M. tuberculosis.

In contrast to the mice whose hematopoietic cells were incapable of responding to IFNγ, mice whose nonhematopoietic cells were IFNγ R1−/− (W K mice) survived the acute phase of 6 the infection (FIG. 3A). They were no less capable of arresting progressive growth of M. tuberculosis in the lungs 4 weeks after infection than were mice with wild type hematopoietic and nonhematopoietic cells (W W mice) (FIG. 3B). In fact, they had significantly fewer bacteria in their lungs at week 3 and 4 (FIG. 4A). Both groups also displayed small pulmonary lesions at that time (FIG. 5). However, the W K mice succumbed earlier than the W W mice during the chronic phase of infection (median survival=16.9±1.1 weeks vs. 27±0.6 weeks, p<0.001). All uninfected bone marrow chimeric mice survived for over 30 weeks, indicating that death of the infected W K mice was due to M. tuberculosis. To assess the cause of death of the W K mice, we examined their ability to control growth of M. tuberculosis and found that as the infection progressed beyond its fourth week, M. tuberculosis continued to grow in the lungs of W K mice while the bacterial burden was maintained at a plateau in W W mice (FIG. 3B). Consequently, the lung bacterial load in W K mice surpassed that in W W mice and by 14 weeks, as the mice of this group began to succumb, the number of mycobacteria in the lungs reached 7.9 0.2 log 10; 1.4 log 10 higher than in W W mice at the same time (p<0.001). The W K mice also had significantly higher bacterial loads in the mediastinal lymph node (p<0.01) (FIG. 4B) and the spleen (p<0.01) (FIG. 4C) on week 14 post-infection. Gross pathologic examination of the lungs showed that W W mice had numerous focal subpleural lesions, whereas W K mice displayed no surface lesions (FIG. 3C). Microscopic examination revealed organized cellular aggregates limited to the peripheral areas of the lungs in W W mice, whereas the entire pulmonary tissue of W K mice was diffusely affected, with a marked increase in cellularity (FIG. 3C). Dense infiltrates of polymorphonuclear granulocytes, most likely neutrophils, were observed in the lungs of W K mice (FIG. 3D). In some areas of the lungs, neutrophils and M. tuberculosis could be visualized in close association (FIG. 3E). Together, these observations indicate that the response to IFNγ in nonhematopoietic cells plays an essential role in the control of M. tuberculosis growth in the lungs after the acute phase of the infection.

Cell Recruitment to the Lungs of IFNγR1 Bone Marrow Chimeras Infected with M. Tuberculosis

To determine whether the susceptibility of W K mice was due to defective activation and/or recruitment of immune cells to the site of infection, we examined the cell populations in the lungs during the course of infection. The total number of cells did not differ significantly between W W and W K mice for up to 9 weeks of infection with M. tuberculosis (FIG. 6A). During this phase, the number of leukocytes in the lungs increased sharply in both groups, peaked at 4 weeks post-infection then decreased progressively until week 9. After week 9, the total number of cells remained unchanged in the lungs of W W mice but increased significantly in W K mice. Analysis of the T cell populations in the lungs of chimeric mice during infection revealed no significant difference in CD4+ or CD8+ T cells between the two groups of mice (FIG. 7A). In both groups of mice, the population of CD4+ T cells increased sharply after infection, peaking at 4 weeks then contracting to a plateau during the chronic phase of infection. The number of CD8+ T cells also increased upon M. tuberculosis infection, peaked at 4 weeks but experienced a smaller decline, without any plateau. As we have previously reported (Wolf et al., 2008), CD4+ and CD8+ T cells also increased in number in the mediastinal (lung-draining) lymph node following infection; there was no significant difference in T cell expansion in the lymph node between W W and W K mice (data not shown).

We (Wolf et al., 2007) and others (Gonzalez-Juarrero et al., 2003; Skold and Behar, 2008) have shown that M. tuberculosis induces the recruitment of several subsets of myeloid cells to the lungs. When we compared populations of myeloid cells in the lungs of M. tuberculosis-infected mice, we found that those of W K mice contained significantly fewer CD11chiCD11bhi myeloid dendritic cells and D11cloCD11bhi inflammatory/interstitial macrophages during the early stages of infection when compared to the lungs of W W mice (FIG. 7B). The differences between the two groups of mice diminished in the later stages of infection, when these myeloid populations contracted in the W W mice. There was no significant difference between the two experimental groups in the populations of CD11chiCD11blo alveolar macrophages and CD11c-CD11bhiGr-1− monocytes. During the acute phase of infection, CD11bhiGr-1hi granulocytes were more abundant in lungs of W K mice than in W W mice (week 3: 5.0 1.2×105 vs. 1.8 0.1×105 cells, p=0.031) but by week 4, this population contracted to the same level as in wild type mice (FIG. 6B). At later time points, flow cytometry analysis confirmed the microscopic findings (FIGS. 3D and 3E): CD11bhiGr-1hi neutrophils were recruited to the lungs in large numbers. By week 9, the abundance of granulocytes rose sharply in the lungs of W K mice whereas it remained unchanged in W W mice (FIG. 6B). At week 14, approximately 2 weeks prior to the average time of death, W K mice had approximately 30% more cells in their lungs than W W mice (1.6 0.17×107 vs. 1.2 0.17×107 cells, p<0.05), and CD11bhiGr-1hi neutrophils fully accounted for this increase (FIGS. 6B and 6C).

Differential Gene Expression in the Lungs of IFNγR Chimeric Mice During M. tuberculosis Infection

To further understand the mechanisms underlying the immune response to M. tuberculosis in W K mice, we compared gene expression profiles in the lungs usingmicroarray analysis. We first focused our attention on well-characterized IFNγ-responsive genes (Ehrt et al., 2001; Sana et al., 2005). During the chronic phase of infection, of the 20 IFN-responsive genes that we selected and analyzed by microarray, twelve were not differentially expressed between the two experimental groups (i.e., less than 2-fold difference), 7 genes were significantly but moderately (more than 2-fold but less than 10-fold difference) underexpressed in the lungs of W K mice in comparison to those of W W mice (FIG. 8). Indoleamine 2,3-dioxygenase (Ido) was the only IFNγ-responsive gene that was underexpressed more than 10-fold: Ido mRNA expression was reduced by 29.4-fold by comparison to W W mice (p=5×10-22) during week 9 post-infection. To confirm these results using an unbiased set of genes, we performed whole genome expression profiling on the lungs of infected chimeric mice. Of 41,171 genes analyzed, 1,761 were underexpressed and 1,894 genes were overexpressed in W K mice compared to W W mice during week 14 post-infection. A large fraction of the underexpressed genes are involved in immune cell recruitment, B cell and NK cell functions and antigen presentation (Table IA), whereas many of the overexpressed genes have roles in tissue remodeling and inflammation (Table IB). At 14 weeks postinfection, Ido remained the most underexpressed gene in the lungs of W K mice infected with M. tuberculosis; its mRNA was expressed at a level 36.6-fold lower than in the lungs of infected W W mice (p=2.4×10-22).

TABLE I
Selected genes underexpressed (A) or overexpressed (B) in the lungs of W Kmice
compared to W W mice 14 weeks post-infection with M. tuberculosis.

| | | | | |
|---|---|---|---|---|
| Name | Sequence Description | Accession # | Fold Change | P-value |

TABLE IA

| | | | | |
|---|---|---|---|---|
| Indo | Indoleamine-pyrrole 2,3 | NM_008324 | −36.59276 | 2E−22 |
| | Dioxygenase | | | |
| Il22 | Interleukin 22 | NM_016971 | −5.74921 | 1.E−06 |
| Igj | Immunoglobulin joining | NM_152839 | −5.08559 | 3.E−15 |
| | chain | | | |
| Klrb1c | Killer cell lectin-like receptor | NM_008527 | −4.87152 | 3.E−14 |
| | subfamily B member 1C | | | |
| Cr2 | Complement receptor 2 | NM_007758 | −4.40919 | 8.E−13 |
| Bank1 | B-cell scaffold protein with | NM_001033350 | −4.16530 | 3.E−05 |
| | ankyrin repeats 1 | | | |
| Igtp | Interferon gamma induced | NM_018738 | −3.44126 | 1.E−11 |
| | GTPase | | | |
| Cd79b | CD79B antigen | NM_008339 | −3.30180 | 3.E−11 |
| H2-DMb2 | Histocompatibility 2, class II, | NM_010388 | −3.18093 | 8.E−11 |
| | locus Mb2 | | | |
| Ighg | Immunoglobulin heavy chain | BC092269 | −3.15632 | 8.E−11 |
| | (gamma polypeptide) | | | |
| Klra10 | Killer cell lectin-like receptor | NM_008459 | −3.10038 | 8.E−09 |
| | subfamily A, member 10 | | | |
| Klra6 | Killer cell lectin-like receptor, | NM_008464 | −3.04309 | 2.E−08 |
| | subfamily A, member 6 | | | |
| Cd19 | CD19 antigen | NM_009844 | −2.88237 | 7.E−10 |
| Trat1 | T cell receptor associated | NM_198297 | −2.77703 | 7.E−09 |
| | transmembrane adaptor 1 | | | |
| Gata3 | GATA binding protein 3 | NM_008091 | −2.73872 | 3.E−09 |
| Cd79a | CD79A antigen | NM_007655 | −2.73226 | 3.E−06 |
| | (immunoglobulin-associated alpha) | | | |
| Klra7 | Killer cell lectin-like receptor, | NM_014194 | −2.71811 | 4.E−09 |
| | subfamily A, member 7 | | | |
| Blr1 | Burkitt lymphoma receptor 1 | NM_007551 | −2.71112 | 3.E−09 |
| Klra8 | Killer cell lectin-like receptor, | NM_010650 | −2.64265 | 9.E−09 |
| | subfamily A, member 8 | | | |
| Ifngr1 | Interferon gamma receptor 1 | NM_010511 | −2.63842 | 6.E−09 |
| C2ta | Class II transactivator | NM_007575 | −2.51527 | 2.E−08 |
| Ccr6 | Chemokine (C-C motif) | NM_009835 | −2.47999 | 3.E−08 |
| | receptor 6] | | | |
| Lta | Lymphotoxin A | NM_010735 | −2.41145 | 7.E−15 |
| Cxcr3 | Mus musculus Chemokine | NM_009910 | −2.40445 | 7.E−08 |
| | (C-X-C motif) receptor 3 | | | |
| Card11 | Caspase recruitment domain | NM_175362 | −2.22414 | 5.E−07 |
| | family, member 11 | | | |
| Stat2 | Signal transducer and activator of | NM_019963 | −2.21540 | 6.E−07 |
| | transcription 2 | | | |
| Tap1 | Transporter 1, ATP-binding cassette, | NM_013683 | −2.12481 | 2.E−06 |
| | sub-family B (MDR/TAP) | | | |
| Stat1 | Signal transducer and activator of | NM_009283 | −2.07716 | 3.E−06 |
| | transcription 1 | | | |
| Nox1 | NADPH oxidase 1 | NM_172203 | −2.04629 | 4.E−01 |
| Tbx21 | T-box 21 | NM_019507 | −2.02845 | 5.E−06 |
| Psmb9 | Proteosome subunit, beta type 9 | NM_013585 | −2.01649 | 6.E−06 |

TABLE IB

| | | | | |
|---|---|---|---|---|
| Stfa1 | Stefin A1 | NM_001001332 | 35.72946 | 3.E−22 |
| Glycam1 | Glycosylation dependent | NM_008134 | 28.69311 | 2.E−20 |
| | cell adhesion molecule 1 | | | |
| Cxcl2 | Chemokine (C-X-C motif) | NM_009140 | 27.19208 | 6.E−22 |
| | ligand 2 | | | |
| Camp | Cathelicidin antimicrobial | NM_009921 | 16.35017 | 1.E−20 |
| | peptide | | | |
| Il17a | Interleukin 17A | NM_010552 | 14.32831 | 2.E−20 |
| Trem1 | Triggering receptor expressed | NM_021406 | 9.14956 | 9.E−19 |
| | on myeloid cells 1 | | | |
| Ccl3 | Chemokine (C-C motif) ligand 3 | NM_011337 | 8.84707 | 1.E−18 |
| Csf3 | Colony stimulating factor 3 | NM_009971 | 6.82582 | 7.E−16 |
| | (granulocyte) | | | |
| Il1r2 | Interleukin 1 receptor, type II | NM_010555 | 6.37657 | 9.E−17 |
| Selp | Selectin, platelet | NM_011347 | 5.72550 | 0.E+00 |
| Sele | Selectin, endothelial cell | NM_011345 | 5.48462 | 9.E−14 |
| Ccr1 | Chemokine (C-C motif) | NM_009912 | 4.77916 | 1.E−06 |
| | receptor 1 | | | |
| Mmp9 | Matrix metallopeptidase 9 | NM_013599 | 3.95812 | 5.E−34 |
| Cxcl4 | Chemokine (C-X-C motif) | NM_019932 | 3.88045 | 7.E−13 |
| | ligand 4 | | | |
| Cxcl5 | Chemokine (C-X-C motif) | NM_009141 | 3.76224 | 1.E−12 |
| | ligand 5 | | | |
| Il23a | Interleukin 23, alpha subunit p19 | NM_031252 | 3.74914 | 4.E−06 |
| Il1b | Interleukin 1 beta | NM_008361 | 3.39765 | 3.E−27 |
| Chi3l1 | Chitinase 3-like 1 | NM_007695 | 2.72644 | 3.E−09 |
| Il17f | Interleukin 17F | NM_145856 | 2.71424 | 1.E−02 |
| Irak2 | Interleukin-1 receptor-associated | NM_172161 | 2.66109 | 5.E−09 |
| | kinase 2 | | | |
| Tnf | Tumor necrosis factor | NM_013693 | 2.60488 | 3.E−17 |
| Il6 | Interleukin 6 | NM_031168 | 2.56139 | 9.E−17 |
| Cxcl1 | Chemokine (C-X-C motif) | NM_008176 | 2.37728 | 9.E−08 |
| | ligand 1 | | | |
| Col15a1 | Procollagen, type XV | NM_009928 | 2.37712 | 1.E−07 |
| Cd14 | CD 14 antigen | NM_009841 | 2.35198 | 1.E−07 |
| Edn1 | Endothelin 1 | NM_010104 | 2.29886 | 2.E−07 |
| Tlr6 | Toll-like receptor 6 | NM_011604 | 2.17960 | 9.E−07 |
| Tlr4 | Toll-like receptor 4 | NM_021297 | 2.05686 | 2.E−10 |

Microarray analysis was conducted on pools of RNA from 5 mice per group and the pools of each group were hybridized against each other. The results are expressed as fold change in mRNA expression.

Characterization of the Expression of Indoleamine 2,3-dioxygenase in the Lungs of IFNγR Chimeric Mice During M. tuberculosis Infection

To confirm the results obtained using whole genome expression profiling by microarray, we first quantified the specific expression of Ido mRNA using quantitative real-time PCR during the course of TB infection. Ido mRNA expression was detected at very low and similar levels in the lungs of W W and W K mice after 2 weeks of infection with M. tuberculosis (FIG. 9A). One week later, Ido expression rose sharply in W W mice and was maintained at a similar level throughout the infection, mirroring the expression of Ifng mRNA in the lungs of these mice (FIG. 9B). In contrast, Ido expression was 30-fold lower in the lungs of mice whose nonhematopoietic cells are unable to respond to IFN, even though the expression of Ifng increased similarly to that in W W mice after week 3 of infection. These findings imply that IFNγ induces Ido expression by nonhematopoietic cells in the lungs during infection with M. tuberculosis, and are consistent with reports that IFNγ can induce Ido expression in cultured fibroblasts (Pfefferkorn et al., 1986) and epithelial cells (Rapoza et al., 1991). We also confirmed that IFNγ treatment of cultured murine NIH/3T3 fibroblasts induces expression of Ido (FIG. 10). We confirmed the underexpression of IDO at the protein level using immunohistochemistry. In the lungs of W W mice during the chronic phase of TB, airway epithelial cells and vascular endothelial cells stained intensely with an antibody to murine IDO.

In addition, IDO expression was detected in cells localized in granulomas that displayed the morphological characteristics of macrophages and/or dendritic cells (FIG. 9C). In comparison, IDO staining in the lungs of W K mice 15 weeks post-infection could only be detected in macrophages and/or dendritic cells in granulomas, without any staining of epithelial or endothelial cells (FIG. 9C). Taken together, these results provide strong evidence that IFNγ-dependent expression of IDO in nonhematopoietic lung cells contributes significantly to the overall expression of IDO in the lungs of mice chronically infected with M. tuberculosis.

In the Absence of IFNγR on Nonhematopoietic Cells, Mice Develop an Excessive IL-17 Response to M. tuberculosis

Whole genome expression profiling of the lungs of chimeric mice chronically infected with M. tuberculosis also revealed overexpression of numerous genes involved in inflammation (Table IB). Since we observed an exuberant inflammatory response, including recruitment of neutrophils, during the chronic stage of infection of W K mice, we characterized the expression of IL-17A in detail, and analyzed the time course of IL17A expression in the lungs of M. tuberculosis-infected mice using quantitative real-time PCR. We also quantitated expression of genes involved in the generation or maintenance of IL-17-secreting cells, i.e. Il6, Tgfb1 and 1123a (Bettelli et al., 2007). The level of expression of Il17a mRNA in the lungs of W W and W K mice was low and equivalent until 4 weeks of infection with M. tuberculosis (FIG. 11A). By week 9 post-infection, less than two weeks before the W K mice started to die, IL17A expression increased more than 10-fold in the lungs of these mice but remained at the same low level in W W mice. By 14 weeks of infection, expression of IL17A in W K mice was 163-fold higher than in W W mice.

Role of Kynurenines in Regulating Development of Th17 Cells

Since we observed marked underexpression of IDO in the lungs of infected W K mice, and since IDO catabolizes tryptophan to products (collectively termed kynurenines) with immunoregulatory properties (Munn and Mellor, 2007), we determined whether defective generation of tryptophan catabolites might account for the overexpression of IL17A in the lungs. First, we examined the effects of tryptophan catabolites on the development of Th17 cells in vitro. An equimolar solution of L-kynurenine, 3′-hydroxy-DL-kynurenine, 3′-hydroxyanthranilic acid, anthranilic acid and quinolinic acid caused dose-dependent inhibition of IL-17 production by CD4+ T cells under Th17 differentiating conditions, which was detectable at a concentration of 7.5 mg/ml; complete inhibition was observed at a concentration of 25 M for the mixture of tryptophan catabolites (FIG. 11B). In these conditions, nonlinear regression analysis revealed an IC50 value of 11.7±1.1 M (10.4±1.1 g/ml). When these compounds were tested individually, 3′-hydroxyanthranilic acid was the most potent inhibitor of IL-17 production, with an IC50 of 27.7±3.7 M, followed by 3′-hydroxy-DL-kynurenine (IC50=68.1±1.4 M) and L kynurenine (IC50=100.8±1.0 M) (Table II). Anthranilic acid and quinolinic acid had no inhibitory effect within the range of concentrations assayed. The observation that the IC50 for the mixture is lower than for any of the individual compounds suggests that two or more of the individual compounds have distinct targets that affect Th17 differentiation and/or IL-17 secretion. We found that kynurenines were able to inhibit IFNγ production during Th1 differentiation but required substantially higher concentrations (Table II): the most potent inhibitor was 3′-hydroxyanthranilic acid, with an IC50 of 57.6±1.0 M. The combination of catabolites did not reduce further the production of IFN (IC50=57.9±1.3 M), therefore, the combination of tryptophan catabolites was approximately 5-fold more potent for inhibition of Th17 versus Th1 differentiation. In light of our observation that W K mice also exhibit poorer control of M. tuberculosis growth late in infection (FIG. 3B), we examined kynurenines for the ability to inhibit growth of M. tuberculosis in vitro (FIG. 10). While we found that kynurenines inhibited growth of M. tuberculosis at concentrations previously reported to inhibit growth of E. coli, the effective concentrations were approximately 20-fold higher than the concentrations that inhibited Th17 differentiation.

Since we also observed that IL-23p19 was overexpressed in the lungs of M. tuberculosis-infected W K mice, and since IL-23 promotes development and maintenance of Th17 cells in peripheral tissues as well as in lymph nodes (McGeachy et al., 2009), we tested the hypothesis that tryptophan catabolites inhibit the action of IL-23 during Th17 differentiation. We confirmed that IL-23 enhanced production of IL-17 under the conditions of our assay, and found that tryptophan catabolites completely abrogated the effect of IL-23, even when they were only included during the latter stages of Th17 differentiation (FIG. 11C).

TABLE II
Table II. 3′-hydroxyanthranilic acid is the most potent tryptophan
catabolite for the inhibition of IL-17 production by Th17 cells in vitro.

IC50(μM)

| Tryptophan metabolites | Th1 | Th17 |
|---|---|---|
| L-Kynurenine | 154.4 ± 1.2 | 100.8 ± 1.0 |
| 3′-Hydroxy-DL-kynurenine | 153.8 ± 1.1 | 68.1 ± 1.4 |
| 3′-Hydroxyanthranilic acid | 57.6 ± 1.0 | 27.7 ± 3.7 |
| Anthranilic acid | No inhibition | No inhibition |
| Quinolinic acid | 111.5 ± 1.4 | No inhibition |
| All | 57.9 ± 1.3 | 11.7 ± 1.1 |

Inhibiting concentrations (IC50) of L-kynurenine, 3′-hydroxy-DL-kynurenine, 3′-hydroxyanthranilic acid, anthranilic acid or quinolinic acid on IFNγ and IL-17 production by differentiating Th1 and Th17 cells in vitro respectively. The results are expressed as the average IC50 values (±S.E.), calculated using a nonlinear regression with variable slope (Prism software, GraphPad).

### Discussion

While IFN is essential for immune control of M. tuberculosis, its targets and functions are still incompletely understood. Since macrophages are thought to be a major cellular reservoir for M. tuberculosis, previous studies have focused on the effects of IFNγ on these cells, and Nos2 and Lrg47/Irgm1 are the only two IFNγ-dependent macrophage effector genes known to be essential for the control of tuberculosis (MacMicking et al., 1997; MacMicking et al., 2003).

In the studies reported here, we provided the first direct evidence that macrophages (and possibly other hematopoietic cells) must be able to respond to IFNγ in vivo in order to control growth of M. tuberculosis during the early, acute stage of infection. However, we also found that nonhematopoietic cells must also be responsive to IFNγ for durable control of bacterial growth and survival of the host. We demonstrated that at least one of the mechanisms involving IFNγ-responsive epithelial and endothelial cells is the expression of IDO and the regulation of IL-17 expression and subsequent neutrophilic inflammation in the lungs.

During the acute stage of infection, the adaptive immune response and its capacity to limit bacterial growth was unaffected by the absence of IFNγR on nonhematopoietic cells. In accord with the observation that IFNγ induces cultured epithelial cells to express the chemokines CXCL9, CXCL10, and CXCL11 (Sauty et al., 1999), we observed a transient deficit in the expression of these IFNγ-inducible chemokines, as well as CCL5 (data not shown), but this had no effect on the recruitment of CD4+ and CD8+ T cells to the lungs in W K mice. The earliest detectable difference between M. tuberculosis-infected W K and W W mice was a defect in recruitment and/or differentiation of myeloid dendritic cells and interstitial macrophages in the lungs. This defect, which was detectable by week 3 postinfection, could actually explain the lower bacterial burden that we consistently observed in the lungs of W K mice at that time. Indeed, a reduced recruitment of potential target cells, such as dendritic cells and macrophages, has been shown to negatively influence the growth of mycobacteria (Davis and Ramakrishnan, 2009). However, this cellular deficiency was a transient phenomenon and by the ninth week of infection, the number of recruited macrophages and myeloid dendritic cells in the lungs was similar between both experimental groups.

Although there were only a few measurable differences in the response to M. tuberculosis in W K compared with W W mice during the early stages of infection, W K mice subsequently exhibited poorer control of bacterial growth and selective overexpression of IL-17 and neutrophilic inflammation in the lungs. These observations could have at least two potential general explanations. One is that nonhematopoietic cells in the lungs are underappreciated as cellular reservoirs of M. tuberculosis, and that IFNγ controls growth of the bacteria that reside in these cells. Despite considerable in vitro data showing that M. tuberculosis can be taken up by cultured epithelial and endothelial cells (Bermudez and Goodman, 1996; Debbabi et al., 2005; Mehta et al., 2006), and some evidence that intact M. tuberculosis can be detected in lung epithelial cells in experimental infections (Rivas-Santiago et al., 2008; Teitelbaum et al., 1999), the majority of the existing evidence strongly favors macrophages and dendritic cells in the lungs as the major cellular reservoirs for M. tuberculosis (Wolf et al., 2007). In our present studies, acid-fast staining of the lungs of W K mice also indicated that most of the bacteria were associated with cells having the morphology of macrophages and/or dendritic cells, and at later stages of infection, bacteria were also associated with neutrophils. Therefore, the in vivo significance of nonhematopoietic cells as reservoirs of M. tuberculosis remains controversial, and it appears unlikely that the course of infection in W K mice was due to uncontrolled replication of M. tuberculosis in lung epithelial or endothelial cells. In light of the evidence that macrophages and dendritic cells are the major reservoirs of M. tuberculosis, any IFNγ-inducible antimycobacterial activity provided by nonhematopoietic cells would likely be achieved through secreted factors. Although nonhematopoietic cells can respond to M. tuberculosis by secreting TNF, GM-CSF, and MCP-1 (CCL2) (Lin et al., 1998) and they can produce numerous antimicrobial products (Evans et al., 2009), few of these are dependent on IFNγ signaling, e.g. inducible nitric oxide synthase, -defensins, NADPH oxidases, and indoleamine 2,3-deoxygenase. Among those, we found that indoleamine 2,3-deoxygenase (IDO) was the most underexpressed IFNγ-responsive gene in W K mice during chronic TB, and that airway epithelial cells and vascular endothelial cells were the major source of this enzyme at that time. IDO catalyzes the first step of the degradation pathway of tryptophan and is highly induced by IFNγ in a wide variety of cells (Carlin et al., 1989). Its antimicrobial activity was originally described as a consequence of tryptophan depletion (Pfefferkorn, 1984). Such mechanism is unlikely to account for our observations, since M. tuberculosis is capable of synthesizing tryptophan (Parish and Stoker, 2002). More recent data indicate that the tryptophan catabolites generated by IDO, or kynurenines, possess broad spectrum bacteriostatic activity (Narui et al., 2009), although we found that inhibition of M. tuberculosis required kynurenine concentrations that are at least 20-fold higher than the concentrations that inhibit Th17 differentiation in vitro. Likewise, since macrophages and dendritic cells in both W W and W K mice are competent to respond to IFNγ and express IDO in vivo (FIG. 9C), it is unlikely that kynurenines mediate antimycobacterial activity strictly in an intracellular environment. Other IFNγ-inducible molecules, expressed in non-hematopoietic cells and with a potential role in bacteriostasis include inducible nitric oxide synthase (NOS2) and members of the -defensin family. The expression level of these genes in the lungs did not suggest any involvement in the mortality experienced by infected W K mice. Lastly, the expression of NADPH oxidase Nox1, also produced by non-hematopoietic cells in response to IFNγ and implicated in the control of bacterial infection (Leto and Geiszt, 2006; Robbins et al., 1994), was only moderately reduced in W K mice. The importance of Nox1 in the control of M. tuberculosis is currently unknown but mice deficient in the closely related phagocyte NADPH oxidase Nox2 do not succumb to TB (Nathan and Shiloh, 2000).

The second general mechanism that may account for the premature death of W K mice involves dysregulation of the immune response during the second month of infection with M. tuberculosis. While recent evidence indicates that IL-10 contributes to regulation of immunity to TB during the chronic stage of infection (Higgins et al., 2009), we did not observe any defect in IL-10 expression in infected W K mice. However, this report emphasizes the importance of immune regulation in the late stage of infection with M. tuberculosis.

Coincidentally, IDO has been increasingly implicated in the regulation of the immune response in cancer, transplantation, autoimmunity, allergies and chronic infections (Brandacher et al., 2008; Katz et al., 2008; Le and Broide, 2006; Zelante et al., 2009). Much attention has been focused on the production of IDO by regulatory dendritic cells (Mellor and Munn, 2004) but several lineages of nonhematopoietic cells derived from human lungs have been reported to regulate T cell proliferation via IDO-dependent mechanisms in vitro (Heseler et al., 2008). In our studies, Ido mRNA was detectable at high levels in the lungs throughout infection with M. tuberculosis but the expression of the protein, as revealed by immunohistochemistry, was limited to myeloid cells during the acute phase (data not shown). Maximum IDO expression appeared after nine weeks of infection, in an IFNγR-dependent fashion and in cells of nonhematopoietic origin, i.e. airway epithelial cells and vascular endothelial cells. This pattern suggests the existence of tissue specific, post-transcriptional regulation mechanisms and it may explain why IDO is only required during the later stages of M. tuberculosis infection. The observation that epithelial and endothelial cells in the lungs of W K did not express detectable amounts of IDO at a time when IDO was readily detected in epithelial and endothelial cells of W W mice provides strong evidence that these nonhematopoietic cells in the lungs were of recipient, and not donor origin. This is consistent with the published observation that bone marrow cells do not differentiate into lung epithelial cells at a detectable frequency (Kotton et al., 2005).

Two other significant observations in chronically infected chimeric mice were the dramatic increase in IL-17 expression, induction of CXCL2, and the massive influx of neutrophils in the lungs, both starting at week 9 and peaking at week 14 of infection. The role of IL-17 on neutrophil recruitment has been extensively studied (Linden et al., 2005), but the relationship between M. tuberculosis infection and IL-17 has only been recently examined (Khader and Cooper, 2008). Most evidence reported to date has indicated a beneficial role for IL-17 in immunity to M. tuberculosis. In particular, IL-17 production is required for a protective memory response following subunit vaccination (Khader et al., 2007). In addition, gene delivery of IL-23 has been shown to positively influence the outcome of M. tuberculosis infection (Happel et al., 2005). One study showed that infection of IFN−/−mice with BCG leads to increased frequency of IL-17 producing cells but the investigators did not report the outcome of this imbalance in terms of pathology or bacterial control (Cruz et al., 2006). In our experiments, given the timing and the amplitude of the neutrophilic inflammation associated with the expression of IL-17 in infected W K mice, it is highly likely that dysregulation of IL-17 expression contributes to the death of W K mice, through extensive tissue damage and subsequent impairment of respiratory function. Unchecked Th17 responses have been associated with several deleterious inflammatory conditions. In particular, a murine model of the severe inflammation observed in chronic granulomatous disease (CGD) patients infected with Aspergillus fumigatus revealed an excessive Th17 response provoked by the impaired conversion of tryptophan into kynurenines (Romani et al., 2008). We found that individual kynurenines have a direct and additive inhibitory effect on the development of Th17 cells in vitro, at concentrations that have been found in vivo during viral pneumonia (Christen et al., 1990). This effect on Th17 differentiation was selective, and at least in part mediated by inhibition of the effects of IL-23; while kynurenines also exerted some inhibitory effect on Th1 differentiation, much higher concentrations were required. We therefore propose a model in which IFN R1−/− nonhematopoietic cells, unable to respond to IFN during the chronic phase of M. tuberculosis infection, fail to express the enzyme IDO and to initiate the conversion of tryptophan to kynurenines. The ongoing bacterial replication characterizing chronic TB in the murine model (Gill et al., 2009) provides sustained pro-inflammatory signals which, in the absence of kynurenines, triggers excessive production of IL-17 and lethal lung neutrophilia.

Since the human response to infection with M. tuberculosis can vary greatly, from asymptomatic latent infection to progressive lung destruction with formation of pulmonary cavities and extensive disability among survivors (de Valliere and Barker, 2004), evidence that defective expression of IDO in pulmonary parenchymal cells, with overexpression of IL-17 and excessive inflammation contributes to fatal infection in mice, provides an opportunity to confirm whether these mechanisms contribute to the morbidity and mortality in humans with tuberculosis.

### EXPERIMENTAL PROCEDURES

### Mice

C57BL/6 congenic CD45.1+ wild type (W) and CD45.2+IFNgR1−/− (K) mice were originally purchased from The Jackson Laboratory (Bar Harbor, Me.). They were bred as homozygotes and maintained under specific pathogen-free conditions at the New York University Medical Center (NYUMC, New York, N.Y.). For infections with Mycobacterium tuberculosis, mice were housed under barrier conditions in the ABSL-3 facility at NYUMC. All mice used were females, between 8 and 12 weeks of age at the beginning of the experiment. For tissue harvest, mice were euthanized by CO2 asphyxiation followed by cervical dislocation. All experiments were performed with the prior approval of NYUMC Institutional Animal Care and Use Committee.

### Generation of Bone Marrow Chimeras

Donor wild type and IFNγR1−/− mice were euthanized using CO2 asphyxiation and cervical dislocation, and femurs and tibias were removed aseptically. Bone marrow was flushed with cold DMEM (Invitrogen), supplemented with 10% heat-inactivated FCS (Invitrogen) and 2 mM L-glutamine (Invitrogen). Cells were washed twice with PBS without calcium and magnesium (Invitrogen), supplemented with 1% FCS. The suspension was depleted of mature T cells by treatment with microbeads coated with anti-Thy 1.2 antibody (Miltenyi Biotec) followed by magnetic activated cell sorting according to the manufacturer's recommendations. Viable cells were counted in a hemacytometer using a Trypan blue exclusion assay. Recipient wild type and IFNgR1−/− mice were irradiated with 10 Gy in split doses at 2-hour intervals. They were reconstituted no later than 6 hours after the last irradiation with 2×106 wild type (W W and W K mice) or IFN R1−/− (K W and K K mice) cells by intravenous injection. Mice were given sulfamethoxazole (150 mg/ml) and trimethoprim (30 mg/ml) in drinking water for the first 3 weeks of reconstitution. Chimeras were used no earlier than 6 weeks after transplantation. Prior to infection with M. tuberculosis, we confirmed that mice reconstituted with congenic bone marrow stem cells had achieved a satisfactory level of chimerism by assessing the number of CD45.1+ (wild type) and CD45.2+ (IFN R1−/−) leukocytes in the lungs, using flow cytometry (FIG. 2). In this organ, the proportion of donor-derived leukocytes averaged 86.6 1% over multiple experiments, after the transfer of either wild type or IFNγ R1−/− marrow, and did not vary over time after M. tuberculosis infection. The same result was found in the spleen (data not shown).

### Bacterial Infection

The H37Rv strain of Mycobacterium tuberculosis was grown as previously described (Banaiee et al., 2006). Chimeras were infected via the aerosol route, using an inhalation exposure system (Glas-Col) (Wolf et al., 2007), calibrated to deliver 150 colony forming units (CFU) per animal. The infectious dose was confirmed on day 1 by plating whole lung homogenates from 5 sentinel mice on Middlebrook 7H11 agar. CFU were counted after incubation at 37° C. for 2-3 weeks. To determine the bacterial load throughout the infection, the right lung, MLN and spleen were harvested from chimeric mice, homogenized and serial dilutions were plated on Middlebrook 7H11 agar.

### Histology and Immunohistochemistry

The left lung was excised, fixed in 4% paraformaldehyde (PFA) for one week at room temperature then embedded in paraffin. Sections of 5 m were cut and stained with hematoxylin and eosin. Alternatively, sections were treated with an acid-fast Kinyoun's stain to reveal the presence of M. tuberculosis and counterstained with Brilliant Green. For indoleamine 2,3-deoxygenase (IDO) immunostaining, PFA-fixed, paraffinembedded lung sections were deparaffinized using CitriSolv solution (Fisher Scientific) and rehydrated by successive baths of decreasing ethanol concentration. Endogenous peroxidase activity was blocked by treatment with 3% H2O2. Endogenous avidin/biotin activity was blocked using a commercially available kit (Vector Laboratories). Non-specific binding sites were saturated by a solution of bovine serum albumin 2%. IDO was stained using a polyclonal rabbit anti-murine IDO antibody (Alexis Biochemicals). Subsequently, the sections were incubated with biotinylated polyclonal goat anti-rabbit IgG, streptavidin-conjugated horseradish peroxidase and DAB'chromogenic substrate (Vector Laboratories). The tissues were counterstained with hematoxylin, dehydrated and mounted using PerMount solution (Fisher Scientific).

Cell isolation and Flow Cytometry

The right lung as well as the MLN were removed and processed for flow cytometry as previously described (Wolf et al., 2007). Antibodies conjugated to various fluorophores and directed against the following surface markers were used: CD4 (RM4-5, BioLegend), CD8 (53-6.7, BD Biosciences), CD62L (MEL-14, BioLegend), CD45RA (14.8, BioLegend), Dx5 (BD Biosciences), B220 (RA3-6B2, BD Biosciences), CD19 (6D5, BioLegend), CD11c (HL3, BD Biosciences), CD11b (M1/70, BD Biosciences), Gr-1 (RB6-8C5, BD Biosciences), CD45.2 (104, BD Biosciences) A minimum of 200,000 events per sample, gated on live cells using forward and side scatter parameters, was acquired using an LSR11 and the FACSDiva software (BD Biosciences). Data were analyzed using the FlowJo software (TreeStar).

### Microarray Analysis

Total RNA was extracted from the left lung using TriZol reagent (Invitrogen) and was processed as previously described for removal of contaminating genomic DNA and reverse transcription (Banaiee et al., 2006). RNA integrity of individual samples was confirmed by ribosomal RNA profiles at the Genomics Facility of the Cancer Institute of NYU Langone Medical Center. RNA samples of each group of mice were pooled and the resulting pools were hybridized against each other for differential gene expression analysis using Agilent Whole Mouse Genome Oligo Microarray (Miltenyi Biotec).

### Real-Time Quantitative RT-PCR

Total RNA was extracted from lung tissues as described above. The cDNA equivalent of 50 ng of total RNA was analyzed for specific gene expression in triplicate for each sample by quantitative real-time PCR using Platinum SYBR Green qPCR SuperMix (Invitrogen) on an MJ Research Opticon 2. Sequences of the primer pairs can be found in Supplementary Table I. Thermal cycling conditions were 95 C for 10 min then 40 cycles at 94 C for 45 s, 58 C for 30 s and 72 C for 30 s. For quantitation, the relative values were determined by comparing the threshold cycle C(t) of each sample to a standard curve consisting of serial dilutions of a positive control cDNA sample. Results were normalized using 18S rRNA expression as an internal standard for each sample.

TABLE III
Primers for quantitative RT-PCR

| | Sequences (5′-3′) | | |
|---|---|---|---|
| | Forward | Amplicon | Tm |
| Genes | Reverse | size | (° C.) |
| Rn18s | GTAACCCGTTGAACCCCATT | 151 | 76.9 |
| | CCATCCAATCGGTAGTAGCG | | |
| Ifng | AGCAACAGCAAGGCGAAAA | 72 | 69.9 |
| | CTGGACCTGTGGGTTGTTGA | | |
| II6 | TAGTCCTTCCTACCCCAATTTCC | 76 | 70.4 |
| | TTGGTCCTTAGCCACTCCTTC | | |
| II17a | GAAGCTCAGTGCCGCCA | 61 | 73.7 |
| | TCATGTGGTGGTCCAGCTTT | | |
| II23a | AGCAACTTCACACCTCCCTAC | 102 | 77.0 |
| | ACTGCTGACTAGAACTCAGGC | | |
| Tgfb1 | TGACGTCACTGGAGTTGTACGG | 170 | 78.1 |
| | GGTTCATGTCATGGATGGTGC | | |
| Ido | ACTGTGTCCTGGCAAACTGGAAG | 141 | 75.8 |
| | AAGCTGCGATTTCCACCAATAGAG | | |

### Cell Culture and Inhibition Studies

For the in vitro differentiation of Th17 cells, CD4+ T cells were isolated from spleen and lymph nodes of C57BL/6 mice followed by magnetic cell sorting using anti-CD4+ antibodycoated microbeads (Miltenyi Biotec). Cells were re-suspended in RPMI supplemented with 10% FBS, hamster anti-murine CD3 antibody (0.25 g/ml), hamster anti-murine CD28 antibody (1 g/ml), anti-murine IL-4 neutralizing antibody (1 g/ml), anti-murine IFNγ neutralizing antibody (1 g/ml), recombinant murine IL-6 (20 ng/ml, PeproTech), recombinant human TGF (0.5 g/ml, PeproTech) and recombinant murine IL-23 (20 ng/ml, eBioscience). All antibodies were purchased from BioLegend. The cells were seeded at a density of 105 cells/well in 96-well plates pre-coated with 0.12 g/ml of goat anti-hamster IgG antibody (Vector Laboratories) and cultured for 6 days at 37° C. in 5% CO2. In some experiments, IL-23 was omitted or only added on the third day of culture. The concentration of IL-17 in culture supernatants was measured by ELISA (R&D Systems).

For comparison studies with Th1 cells, T cells were differentiated in the same in vitro conditions, with only anti-murine IL-4 neutralizing antibody, recombinant murine IL-2 (10 ng/ml, eBioscience) and recombinant murine IL-12p70 (20 ng/ml, BD Biosciences) and CD3/CD28 stimulation. The concentration of IFN in culture supernatants was measured by ELISA (BD Biosciences). L-kynurenine, 3′-hydroxy-DL-kynurenine, 3′-hydroxyanthranilic acid, anthranilic acid and quinolinic acid (Sigma) were dissolved in RPMI medium under agitation at 1 mM concentration and sterile filtered. Tryptophan metabolites were added individually or in combination, in twofold dilutions, to the differentiating medium of Th1 or Th17 cells for the 6 days of culture.

### Statistical Analysis

Results are expressed as mean and standard error. Student's two-tailed t-test was used to compare experimental groups, unless otherwise stated, with P<0.05 considered significant.

### REFERENCES

Banaiee, N., Kincaid, E. Z., Buchwald, U., Jacobs, W. R., Jr., and Ernst, J. D. (2006). Potent inhibition of macrophage responses to IFN-gamma by live virulent Mycobacterium tuberculosis is independent of mature mycobacterial lipoproteins but dependent on TLR2. J Immunol 176, 3019-3027.
Bermudez, L. E., and Goodman, J. (1996). Mycobacterium tuberculosis invades and replicates within type II alveolar cells. Infection and immunity 64, 1400-1406.
Bettelli, E., Oukka, M., and Kuchroo, V. K. (2007). T(H)-17 cells in the circle of immunity and autoimmunity. Nature immunology 8, 345-350.
Brandacher, G., Margreiter, R., and Fuchs, D. (2008). Clinical relevance of indoleamine 2,3-dioxygenase for alloimmunity and transplantation. Curr Opin Organ Transplant 13, 10-15.
Carlin, J. M., Ozaki, Y., Byrne, G. I., Brown, R. R., and Borden, E. C. (1989). Interferons and indoleamine 2,3-dioxygenase: role in antimicrobial and antitumor effects. Experientia 45, 535-541.
Christen, S., Peterhans, E., and Stocker, R. (1990). Antioxidant activities of some tryptophan metabolites: possible implication for inflammatory diseases. Proc Natl Acad Sci USA 87, 2506-2510.
Cooper, A. M., Dalton, D. K., Stewart, T. A., Griffin, J. P., Russell, D. G., and Orme, I. M. (1993). Disseminated tuberculosis in interferon gamma gene-disrupted mice. The Journal of experimental medicine 178, 2243-2247.
Cruz, A., Khader, S. A., Torrado, E., Fraga, A., Pearl, J. E., Pedrosa, J., Cooper, A. M., and
Castro, A. G. (2006). Cutting edge: IFN-gamma regulates the induction and expansion of IL-17-producing CD4 T cells during mycobacterial infection. J Immunol 177, 1416-1420.
Davis, J. M., and Ramakrishnan, L. (2009). The role of the granuloma in expansion and dissemination of early tuberculous infection. Cell 136, 37-49.
de Valliere, S., and Barker, R. D. (2004). Residual lung damage after completion of treatment for multidrug-resistant tuberculosis. Int J Tuberc Lung Dis 8, 767-771.
Debbabi, H., Ghosh, S., Kamath, A. B., Alt, J., Demello, D. E., Dunsmore, S., and Behar, S. M. (2005). Primary type II alveolar epithelial cells present microbial antigens to antigen-specific CD4+ T cells. Am J Physiol Lung Cell Mol Physiol 289, L274-279.
Ehrt, S., Schnappinger, D., Bekiranov, S., Drenkow, J., Shi, S., Gingeras, T. R., Gaasterland, T., Schoolnik, G., and Nathan, C. (2001). Reprogramming of the macrophage transcriptome in response to interferon-gamma and Mycobacterium tuberculosis: signaling roles of nitric oxide synthase-2 and phagocyte oxidase. The Journal of experimental medicine 194, 1123-1140.
Evans, S. E., Scott, B. L., Clement, C. G., Larson, D. T., Kontoyiannis, D., Lewis, R. E., Lasala, P. R., Pawlik, J., Peterson, J. W., Chopra, A. K., et al. (2009). Stimulated Innate Resistance of Lung Epithelium Protects Mice Broadly Against Bacteria and Fungi. American journal of respiratory cell and molecular biology.
Faliarino, F., Grohmann, U., Vacca, C., Bianchi, R., Orabona, C., Spreca, A., Fioretti, M. C., and Puccetti, P. (2002). T cell apoptosis by tryptophan catabolism. Cell Death and Differentiation 9: 1069-1077.
Fieschi, C., Dupuis, S., Catherinot, E., Feinberg, J., Bustamante, J., Breiman, A., Altare, F., Baretto, R., Le Deist, F., Kayal, S., et al. (2003). Low penetrance, broad resistance, and favorable outcome of interleukin 12 receptor beta 1 deficiency: medical and immunological implications. The Journal of Experimental Medicine 197, 527-535.
Filipe-Santos, O., Bustamante, J., Chapgier, A., Vogt, G., de Beaucoudrey, L., Feinberg, J., Jouanguy, E., Boisson-Dupuis, S., Fieschi, C., Picard, C., and Casanova, J. L. (2006). Inborn errors of IL-12/23- and IFN-gamma-mediated immunity: molecular, cellular, and clinical features. Seminars in immunology 18, 347-361.
Flynn, J. L., Chan, J., Triebold, K. J., Dalton, D. K., Stewart, T. A., and Bloom, B. R. (1993). An essential role for interferon gamma in resistance to Mycobacterium tuberculosis infection. The Journal of experimental medicine 178, 2249-2254.
Gill, W. P., Harik, N. S., Whiddon, M. R., Liao, R. P., Mittler, J. E., and Sherman, D. R. (2009). A replication clock for Mycobacterium tuberculosis. Nat Med 15, 211-214.
Gonzalez-Juarrero, M., Shim, T. S., Kipnis, A., Junqueira-Kipnis, A. P., and Orme, I. M. (2003). Dynamics of macrophage cell populations during murine pulmonary tuberculosis. J Immunol 171, 3128-3135.
Gutierrez, M. G., Master, S. S., Singh, S. B., Taylor, G. A., Colombo, M. I., and Deretic, V. (2004). Autophagy is a defense mechanism inhibiting BCG and Mycobacterium tuberculosis survival in infected macrophages. Cell 119, 753-766.
Happel, K. I., Lockhart, E. A., Mason, C. M., Porretta, E., Keoshkerian, E., Odden, A. R., Nelson, S., and Ramsay, A. J. (2005). Pulmonary interleukin-23 gene delivery increases local T-cell immunity and controls growth of Mycobacterium tuberculosis in the lungs. Infection and immunity 73, 5782-5788.
Heseler, K., Spekker, K., Schmidt, S. K., MacKenzie, C. R., and Daubener, W. (2008). Antimicrobial and immunoregulatory effects mediated by human lung cells: role of IFN-gammainduced tryptophan degradation. FEMS immunology and medical microbiology 52, 273-281.
Higgins, D. M., Sanchez-Campillo, J., Rosas-Taraco, A. G., Lee, E. J., Orme, I. M., and Gonzalez-Juarrero, M. (2009). Lack of IL-10 alters inflammatory and immune responses during pulmonary Mycobacterium tuberculosis infection. Tuberculosis (Edinb) 89, 149-157.
Jouanguy, E., Lamhamedi-Chemadi, S., Altare, F., Fondaneche, M. C., Tuerlinckx, D., Blanche, S., Emile, J. F., Gaillard, J. L., Schreiber, R., Levin, M., et al. (1997). Partial interferon-gamma receptor 1 deficiency in a child with tuberculoid bacillus Calmette-Guerin infection and a sibling with clinical tuberculosis. The Journal of clinical investigation 100, 2658-2664.
Katz, J. B., Muller, A. J., and Prendergast, G. C. (2008). Indoleamine 2,3-dioxygenase in T-cell tolerance and tumoral immune escape. Immunol Rev 222, 206-221.
Khader, S. A., Bell, G. K., Pearl, J. E., Fountain, J. J., Rangel-Moreno, J., Cilley, G. E., Shen, F., Eaton, S. M., Gaffen, S. L., Swain, S. L., et al. (2007). IL-23 and IL-17 in the establishment of protective pulmonary CD4+ T cell responses after vaccination and during Mycobacterium tuberculosis challenge. Nature immunology 8, 369-377.
Khader, S. A., and Cooper, A. M. (2008). IL-23 and IL-17 in tuberculosis. Cytokine 41, 79-83.
Kotton, D. N., Fabian, A. J., and Mulligan, R. C. (2005). Failure of bone marrow to reconstitute lung epithelium. American journal of respiratory cell and molecular biology 33, 328-334.
Le, A. V., and Broide, D. H. (2006). Indoleamine-2,3-dioxygenase modulation of allergic immune responses. Curr Allergy Asthma Rep 6, 27-31.
Leto, T. L., and Geiszt, M. (2006). Role of Nox family NADPH oxidases in host defense. Antioxid Redox Signal 8, 1549-1561.
Lin, Y., Zhang, M., and Barnes, P. F. (1998). Chemokine production by a human alveolar epithelial cell line in response to Mycobacterium tuberculosis. Infection and immunity 66, 1121-1126.
Linden, A., Laan, M., and Anderson, G. P. (2005). Neutrophils, interleukin-17A and lung disease. Eur Respir J 25, 159-172.
MacMicking, J. D., North, R. J., LaCourse, R., Mudgett, J. S., Shah, S. K., and Nathan, C. F. (1997). Identification of nitric oxide synthase as a protective locus against tuberculosis. Proceedings of the National Academy of Sciences of the United States of America 94, 5243-5248.
MacMicking, J. D., Taylor, G. A., and McKinney, J. D. (2003). Immune control of tuberculosis by IFN-gamma-inducible LRG-47. Science (New York, N.Y. 302, 654-659.
McGeachy, M. J., Chen, Y., Tato, C. M., Laurence, A., Joyce-Shaikh, B., Blumenschein, W. M., McClanahan, T. K., O'Shea, J. J., and Cua, D. J. (2009). The interleukin 23 receptor is essential for the terminal differentiation of interleukin 17-producing effector T helper cells in vivo. Nature immunology 10, 314-324.
Mehta, P. K., Karls, R. K., White, E. H., Ades, E. W., and Quinn, F. D. (2006). Entry and intracellular replication of Mycobacterium tuberculosis in cultured human microvascular endothelial cells. Microb Pathog 41, 119-124.
Mellor, A. L., and Munn, D. H. (2004). IDO expression by dendritic cells: tolerance and tryptophan catabolism. Nat Rev Immunol 4, 762-774.
Munn, D. H., and Mellor, A. L. (2007). Indoleamine 2,3-dioxygenase and tumor-induced tolerance. The Journal of clinical investigation 117, 1147-1154.
Nagabhushanam, V., Solache, A., Ting, L. M., Escaron, C. J., Zhang, J. Y., and Ernst, J. D. (2003). Innate inhibition of adaptive immunity: Mycobacterium tuberculosis-induced IL-6 inhibits macrophage responses to IFN-gamma. J Immunol 171, 4750-4757.
Narui, K., Noguchi, N., Saito, A., Kakimi, K., Motomura, N., Kubo, K., Takamoto, S., and Sasatsu, M. (2009). Anti-infectious activity of tryptophan metabolites in the L-tryptophan-Lkynurenine pathway. Biol Pharm Bull 32, 41-44.
Nathan, C., and Shiloh, M. U. (2000). Reactive oxygen and nitrogen intermediates in the relationship between mammalian hosts and microbial pathogens. Proceedings of the National Academy of Sciences of the United States of America 97, 8841-8848.
Parish, T., and Stoker, N. G. (2002). The common aromatic amino acid biosynthesis pathway is essential in Mycobacterium tuberculosis. Microbiology 148, 3069-3077.
Pfefferkorn, E. R. (1984). Interferon gamma blocks the growth of Toxoplasma gondii in human fibroblasts by inducing the host cells to degrade tryptophan. Proceedings of the National Academy of Sciences of the United States of America 81, 908-912.
Pfefferkom, E. R., Rebhun, S., and Eckel, M. (1986). Characterization of an indoleamine 2,3-dioxygenase induced by gamma-interferon in cultured human fibroblasts. J Interferon Res 6, 267-279.
Rapoza, P. A., Tahija, S. G., Carlin, J. P., Miller, S. L., Padilla, M. L., and Byrne, G. I. (1991). Effect of interferon on a primary conjunctival epithelial cell model of trachoma. Invest Ophthalmol V is Sci 32, 2919-2923.
Rivas-Santiago, B., Contreras, J. C., Sada, E., and Hernandez-Pando, R. (2008). The potential role of lung epithelial cells and beta-defensins in experimental latent tuberculosis. Scandinavian journal of immunology 67, 448-452.
Robbins, R. A., Springall, D. R., Warren, J. B., Kwon, O. J., Buttery, L. D., Wilson, A. J., Adcock, I. M., Riveros-Moreno, V., Moncada, S., Polak, J., and et al. (1994). Inducible nitric oxide synthase is increased in murine lung epithelial cells by cytokine stimulation. Biochemical and biophysical research communications 198, 835-843.
Romani, L., Fallarino, F., De Luca, A., Montagnoli, C., D'Angelo, C., Zelante, T., Vacca, C., Bistoni, F., Fioretti, M. C., Grohmann, U., et al. (2008). Defective tryptophan catabolism underlies inflammation in mouse chronic granulomatous disease. Nature 451, 211-215.
Sana, T. R., Janatpour, M. J., Sathe, M., McEvoy, L. M., and McClanahan, T. K. (2005). Microarray analysis of primary endothelial cells challenged with different inflammatory and immune cytokines. Cytokine 29, 256-269.
Sauty, A., Dziejman, M., Taha, R. A., Iarossi, A. S., Neote, K., Garcia-Zepeda, E. A., Hamid, Q., and Luster, A. D. (1999). The T cell-specific CXC chemokines IP-10, Mig, and I-TAC are expressed by activated human bronchial epithelial cells. J Immunol 162, 3549-3558.
Schaible, U. E., Sturgill-Koszycki, S., Schlesinger, P. H., and Russell, D. G. (1998). Cytokine activation leads to acidification and increases maturation of Mycobacterium avium-containing phagosomes in murine macrophages. J Immunol 160, 1290-1296.
Schroder, K., Hertzog, P. J., Ravasi, T., and Hume, D. A. (2004). Interferon-gamma: an overview of signals, mechanisms and functions. J Leukoc Biol 75, 163-189.
Skold, M., and Behar, S. M. (2008). Tuberculosis triggers a tissue-dependent program of differentiation and acquisition of effector functions by circulating monocytes. J Immunol 181, 6349-6360.
Teitelbaum, R., Schubert, W., Gunther, L., Kress, Y., Macaluso, F., Pollard, J. W., McMurray, D. N., and Bloom, B. R. (1999). The M cell as a portal of entry to the lung for the bacterial pathogen Mycobacterium tuberculosis. Immunity 10, 641-650.
Terness, P., Bauer, T. M., Rose, L., Dufter, C., Watzlik, A., Simon, H., and Opelz, G. (2002). Inhibition of allogeneic T cell proliferation by Indoleamine 2,3-dioxygenase expressing dendritic cells: mediation of suppression by tryptophan metabolites. The Journal of Experimental Medicine 196, 447-457.
Ting, L. M., Kim, A. C., Cattamanchi, A., and Ernst, J. D. (1999). Mycobacterium tuberculosis inhibits IFN-gamma transcriptional responses without inhibiting activation of STAT1. J Immunol 163, 3898-3906.
Wolf, A. J., Desvignes, L., Linas, B., Banaiee, N., Tamura, T., Takatsu, K., and Ernst, J. D. (2008). Initiation of the adaptive immune response to Mycobacterium tuberculosis depends on antigen production in the local lymph node, not the lungs. The Journal of experimental medicine 205, 105-115.
Wolf, A. J., Linas, B., Trevejo-Nunez, G. J., Kincaid, E., Tamura, T., Takatsu, K., and Ernst, J. D. (2007). Mycobacterium tuberculosis infects dendritic cells with high frequency and impairs their function in vivo. J Immunol 179, 2509-2519.
Yap, G. S., and Sher, A. (1999). Effector cells of both nonhemopoietic and hemopoietic origin are required for interferon (IFN)-gamma- and tumor necrosis factor (TNF)-alpha-dependent host resistance to the intracellular pathogen, Toxoplasma gondii. The Journal of experimental medicine 189, 1083-1092.
Zelante, T., Fallarino, F., Bistoni, F., Puccetti, P., and Romani, L. (2009). Indoleamine 2,3-dioxygenase in infection: the paradox of an evasive strategy that benefits the host. Microbes Infect 11, 133-141.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for modulating IL-17 expressing cells comprising contacting T cells with one or more kynurenine, tryptophan metabolite, or analog or antagonist thereof.

2. The method of claim 1 wherein the differentiation, activation or activity of IL-17 expressing Th17 cells are inhibited by contacting Th17 cells with one or more kynurenine, tryptophan metabolite, or analog thereof.

3. The method of claim 2 wherein IL-17 expression is reduced.

4. The method of claim 1 wherein the one or more kynurenine or tryptophan metabolite is L-kynurenine, 3-hydroxy-DL-kynurenine, and/or 3-hydroxyanthranilic acid.

5. A method for assaying for drug candidate compounds that modulate Th17 cells comprising contacting one or more candidate compound with Th17 cells or a cellular sample including Th17 cells, in the presence or absence of one or more kynurenine, under conditions that allow said compound to act on or come in contact with the cells, and detecting the activity of the Th17 cells or of the kynurenine.

6. The method of claim 5 said method comprising:
(a) contacting a population of mammalian cells including Th17 cells with one or more compound that exhibits kynurenine activity or a candidate compound to determine its kynurenine-like activity, and
(b) measuring a property related to Th17 cell activity, differentiation, or proliferation.

7. The method of claim wherein the property measured is the Th17 cell activity of expression of an interleukin.

8. The method of claim wherein the property measured is the Th17 cell activity of expression of IL-17.

9. A method or assay system for screening potential drugs effective to mimic or inhibit physiological kynurenines wherein one or more potential drug are introduced into a test system containing Th17 cells, in the presence or absence of one or more known kynurenine, and the system thereafter examined to observe any changes in the activity of the Th17 cells or in the amounts of a Th17 cell expressed factor, due either to the addition of the potential drug alone, or due to the effect of said drug on added quantities of the known kynurenine.

10. The method of claim 9 whereby the activity of Th17 cells is measured via an inflammatory response.

11. The method of claim 9 whereby the activity of Th17 cells is measured by assessing the amount of IL-17 expressed in the test system.

12. A test kit for the determination of the presence of Th17 cells or the quantitative analysis of Th17 cells or their activity in a sample comprising one or more kynurenine and a labeled component which is a binding partner to a Th17 cell marker or Th17 cell expressed factor, whereby the presence of Th17 cells or the quantitative analysis of Th17 cells or their activity is determined by assessing the amount of labeled component which is bound to the Th17 cells or the Th17 cell expressed factor in the sample.

13. The test kit of claim 12 wherein the labeled component is a binding partner to IL-17.

14. A method for modulating Th17 cells in an animal comprising administering to said animal an effective amount of one or more tryptophan metabolite, kynurenine, or kynurenine analog or antagonist to said mammal, whereby the activity of said Th17 cells is altered.

15. The method of claim 14 whereby Th17 cells are inhibited by administering one or more kynurenine or kynurenine analog.

16. The method of claim 15 wherein the one or more kynurenine is selected from L-kynurenine, 3-hydroxy-DL-kynurenine, and/or 3-hydroxyanthranilic acid.

17. A method for modulating inflammation or an inflammatory response in an animal comprising administering to said animal an effective amount of a tryptophan metabolite, kynurenine, or kynurenine analog or antagonist to said mammal, whereby the amount or extent of inflammation or the inflammatory response is altered.

18. The method of claim 17 whereby Th17 cells are inhibited by administering one or more kynurenine or kynurenine analog.

19. The method of claim 18 wherein the one or more kynurenine is selected from L-kynurenine, 3-hydroxy-DL-kynurenine, and/or 3-hydroxyanthranilic acid.

20. A method for treating or alleviating an autoimmune disease or disorder in an animal comprising administering to said animal an effective amount of a tryptophan metabolite, kynurenine, or kynurenine analog to said mammal, whereby a physiologically relevant aspect of the autoimmune disease or disorder is reduced.

21. The method of claim wherein the one or more kynurenine is selected from L-kynurenine, 3-hydroxy-DL-kynurenine, and/or 3-hydroxyanthranilic acid.


SEQUENCE LISTING (14 sequences)

SEQ ID NO: 1 - DNA, 20 bp, Artificial Sequence
  Forward primer
gtaacccgttgaaccccatt

SEQ ID NO: 2 - DNA, 20 bp, Artificial Sequence
  Reverse primer
ccatccaatcggtagtagcg

SEQ ID NO: 3 - DNA, 19 bp, Artificial Sequence
  Forward primer
agcaacagcaaggcgaaaa

SEQ ID NO: 4 - DNA, 20 bp, Artificial Sequence
  Reverse primer
ctggacctgtgggttgttga

SEQ ID NO: 5 - DNA, 23 bp, Artificial Sequence
  Forward primer
tagtccttcctaccccaatttcc

SEQ ID NO: 6 - DNA, 21 bp, Artificial Sequence
  Reverse primer
ttggtccttagccactccttc

SEQ ID NO: 7 - DNA, 17 bp, Artificial Sequence
  Forward primer
gaagctcagtgccgcca

SEQ ID NO: 8 - DNA, 20 bp, Artificial Sequence
  Reverse primer
tcatgtggtggtccagcttt

SEQ ID NO: 9 - DNA, 21 bp, Artificial Sequence
  Forward primer
agcaacttcacacctccctac

SEQ ID NO: 10 - DNA, 21 bp, Artificial Sequence
  Reverse primer
actgctgactagaactcaggc

SEQ ID NO: 11 - DNA, 22 bp, Artificial Sequence
  Forward primer
tgacgtcactggagttgtacgg

SEQ ID NO: 12 - DNA, 21 bp, Artificial Sequence
  Reverse primer
ggttcatgtcatggatggtgc

SEQ ID NO: 13 - DNA, 23 bp, Artificial Sequence
  Forward primer
actgtgtcctggcaaactggaag

SEQ ID NO: 14 - DNA, 24 bp, Artificial Sequence
  Reverse primer
aagctgcgatttccaccaatagag